(19) United States
(12) Patent Application Publication
(10) Pub. No.: US 2026/0182570 A1
(43) Pub. Date: Jul. 2, 2026

(54) DUAL MODE OF ACTION NATURAL PRODUCT-BASED PROHERBICIDES AND USES THEREOF

(71) Applicants: The United States of America, as represented by the Secretary of Agriculture, Washington, DC (US); UNIVERSITY OF MISSISSIPPI, University, MS (US)

(72) Inventors: JOANNA N. BAJSA-HIRSCHEL, OXFORD, MS (US); AMAR G. CHITTIBOYINA, OXFORD, MS (US); SHAMBA CHATTERJEE, OXFORD, MS (US); STEPHEN O. DUKE, OXFORD, MS (US)

(21) Appl. No.: 19/424,296

(22) Filed: Dec. 18, 2025

Related U.S. Application Data
(60) Provisional application No. 63/739,929, filed on Dec. 30, 2024.

(51) Int. Cl. A01N 43/32; A01N 25/02; C07D 319/06; C07D 319/08

(52) U.S. Cl. CPC A01N 43/32; A01N 25/02; C07D 319/06; C07D 319/08

(57) ABSTRACT

Disclosed are dual mode of action (DMOA) natural product-based proherbicide compounds or herbicidally-effective salts thereof, compositions comprising such DMOA proherbicide compounds or herbicidally-effective salts thereof; methods of preparing such DMOA proherbicide compounds, herbicidally-effective salts thereof, and/or compositions; and methods for controlling undesired vegetation by contacting the undesired vegetation or its environment with an effective amount of such a DMOA proherbicide compound, herbicidally-effective salt thereof, or composition.

### CROSS-REFERENCE TO RELATED APPLICATIONS

This application claims priority benefit from U.S. Provisional Application No. 63/739,929, filed Dec. 30, 2024. The contents of this patent application are hereby expressly incorporated by reference in their entirety.

### FIELD OF THE DISCLOSURE

The disclosure relates generally to the field of weed management. More specifically, to novel dual mode of action (DMOA) proherbicide compounds and compositions containing such compounds for the control of undesired vegetation.

### BACKGROUND

The selective control of unwanted vegetation, such as weeds, is a major industry. The aim of using herbicides is to improve agricultural productivity by killing unwanted herbs and weeds. Vegetation can be controlled using herbicides that are pre-emergent or post-emergent. Pre-emergent herbicides, by definition, are herbicides that are applied prior to the emergence of weeds above the ground. Post-emergent herbicides are used to kill weeds after they have emerged above the ground

The use of some synthetic herbicides has led to both human and environmental toxicological issues. These problems have led to stringent regulations on the use of herbicides, which led to increases in the demand for bio-based herbicides. At present, bialaphos, phosphinothricin, pelargonic acid, and triketone herbicides are among the few commercially-available bio-based herbicides. Triketone herbicides, for example, are 4-hydroxyphenylpyruvate dioxygenase (HPPD)-inhibiting bio-based herbicides.

Furthermore, to date, weeds have evolved resistance to most herbicides, including the vast majority of known commercial herbicide sites of action. Development of new bio-based herbicides is a key part of combating herbicide resistance.

Thus, new bio-based herbicides that are safe for humans, animals, and the environment are urgently needed.

### SUMMARY

Disclosed are a series of structurally related triketone and keto-diester herbicides that are 4-hydroxyphenylpyruvate dioxygenase (HPPD) inhibitors and that are linked to a fatty acid having 1 to 20 carbon atoms, such as the natural herbicide, pelargonic acid. These molecules are designed to break down into the fatty acid (e.g., pelargonic acid) and the HPPD inhibitor once systemically absorbed by the plant. Herbicides that are not in the active form until chemically altered in the plant are termed proherbicides. There are fourteen commercial HPPD inhibitor herbicides, and six of these are proherbicides. However, all of these proherbicides are activated to only one herbicide, a HPPD inhibitor. The new chemical entities disclosed herein breakdown into two types of herbicides, a HPPD inhibitor and, for example, a membrane disruptor in the plant. These proherbicide compounds are not in scientific or patent literature. These new chemical entities, including triketone or keto-diester HPPD inhibitor herbicides linked to pelargonic acid (or other fatty acid having 1 to 20 carbon atoms), provide a new class of herbicides to effectively manage weed control in commercial crops. New herbicide classes are badly needed because of the rapid evolution of both target-site and non-target-site based herbicide resistance in weeds. These new compounds can be used for both weed and herbicide resistance management. The tethering of a fatty acid to an HPPD-based herbicide is expected to confer a dual mechanism of action and improve the absorption, permeability, and overall physicochemical properties of the resulting proherbicide. Their dual mode of action (DMOA) capability makes evolution of resistance in weeds less likely. Another possible use is as insecticides on blood-feeding insects (e.g., ticks, mosquitos, and bed bugs), as HPPD inhibitors have recently been found to kill these insects after a blood meal because the insects' HPPD is needed to detoxify tyrosine from blood.

Provided herein are novel DMOA proherbicide compounds, methods of making such compounds, biocontrol compositions comprising such compounds, and methods of using such compounds and compositions as herbicides.

In an embodiment, the disclosure relates to a proherbicide compound having formula (IX)

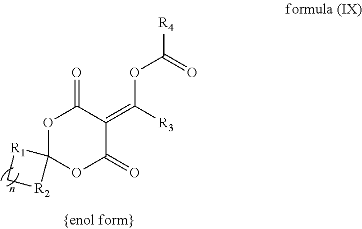

or a herbicidally-effective salt thereof,
wherein n is independently 0, 1, 2, 3, 4, or 5; R₁ and R₂ are independently H or an alkyl group;
R₄ is independently an alkyl chain having 1 to 20 carbon atoms, including, but not limited to alkyl chain with terminal hydroxy or halogen groups; and R₃ is independently

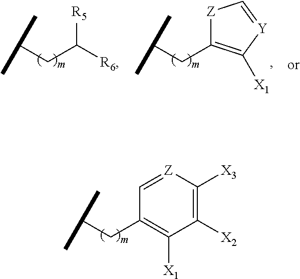

wherein m is independently 0, 1, 2, 3, 4, or 5; R₅ and R₆ are independently H, alkyl, aryl, heteroaryl, alkyl aryl, or alkyl heteroaryl; X₁, X₂, and X₃ are independently H, F, Cl, Br, I, NO₂, SCH₃, SO₂CH₃, or CH₂OR₇, wherein R₇ is independently any alkyl chain or halogenated alkyl chain; Y is independently NH, O, or S; and Z is independently CH or N.

In an embodiment, the disclosure relates to a method for preparing the proherbicide compound having formula (IX), above, or a herbicidally effective salt thereof, wherein the method comprises condensation of at least one tautomer of a HPPD inhibitor of formula (VIII)

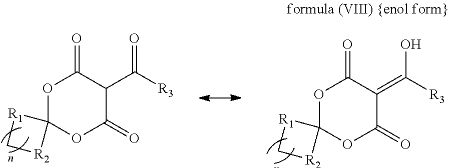

with at least one fatty acid having 6 to 20 carbon atoms including fatty acids with terminal functionalization in the presence of a base and an organic solvent in a single pot reaction. In formula (VIII), n is independently 0, 1, 2, 3, 4, or 5; R₁ and R₂ are independently H or an alkyl group; and R₃ is independently

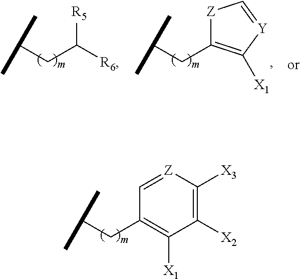

wherein m is independently 0, 1, 2, 3, 4, or 5; R₅ and R₆ are independently H, alkyl, aryl, heteroaryl, alkyl aryl, or alkyl heteroaryl; X₁, X₂, and X₃ are independently H, F, Cl, Br, I, NO₂, SCH₃, SO₂CH₃, or CH₂OR₇, wherein R₇ is independently any alkyl chain or halogenated alkyl chain; Y is independently NH, O, or S; and Z is independently CH or N. The base may be, for example, triethyl amine. The organic solvent may be, for example, 1,2-dichloroethane.

In an embodiment, the disclosure relates to a proherbicide compound having at least one tautomer of formula (XI)

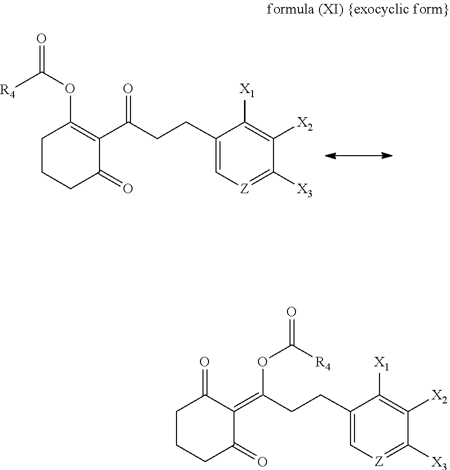

or a herbicidally-effective salt thereof,
wherein R₄ is independently an alkyl chain having 1 to 20 carbon atoms, including, but not limited to alkyl chain with terminal hydroxy or halogen groups; X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently CH or N.

In an embodiment, the disclosure relates to a method for preparing the proherbicide compound having formula (XI), above, or a herbicidally effective salt thereof, wherein the method comprises condensation of at least one tautomer of a HPPD inhibitor of formula (X)

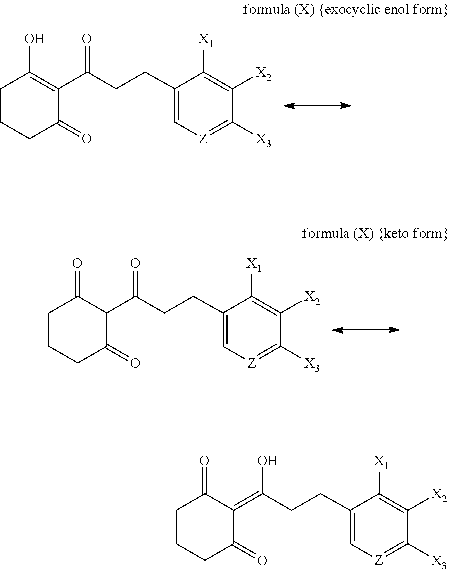

with at least one fatty acid having 6 to 20 carbon atoms including fatty acids with terminal functionalization in the presence of a base and an organic solvent in a single pot reaction. In formula (X), X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently CH or N. The base may be, for example, triethyl amine. The organic solvent may be, for example, 1,2-dichloroethane.

In an embodiment, the disclosure relates to a proherbicide compound having at least one tautomer of formula (XIII)

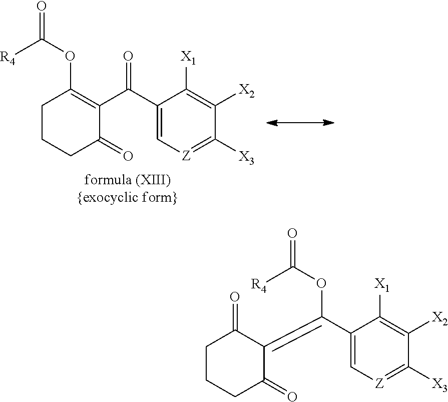

or a herbicidally-effective salt thereof,
wherein R₄ is independently an alkyl chain having 1 to 20 carbon atoms, including, but not limited to alkyl chain with hydroxy or halogen groups; X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently N; or
wherein R₄ is independently an alkyl chain having 7 to 20 carbon atoms, including, but not limited to alkyl chain with hydroxy or halogen groups; X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently CH or N; or
wherein R₄ is independently an alkyl chain having 1 to 20 carbon atoms, including, but not limited to alkyl chain with hydroxy or halogen groups; X₁, X₂, and X₃ are independently H, F, Cl, Br, I, NO₂, SCH₃, SO₂CH₃, or CH₂OR₇, wherein R₇ is independently any alkyl chain or functionalized alkyl chain, and wherein at least one of X₁, X₂, and X₃ is CH₂OR₇; and Z is independently CH or N.

In an embodiment, the disclosure relates to a method for preparing the proherbicide compound having formula (XIII), above, or a herbicidally effective salt thereof, wherein the method comprises condensation of at least one tautomer of a HPPD inhibitor of formula (XII)

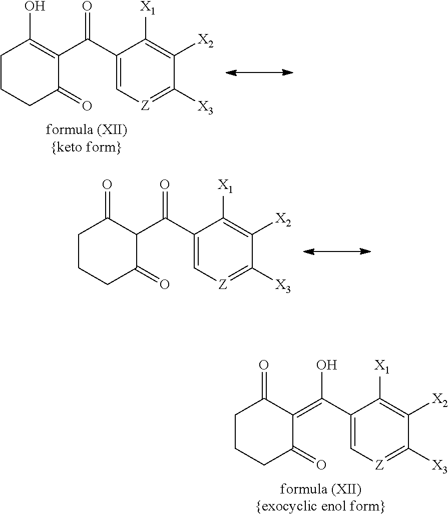

with at least one fatty acid or functionalized fatty acid having 6 to 20 carbon atoms in the presence of a base and a non-protic organic solvent in a single pot reaction to prepare a novel DMOA proherbicide with two independent mechanisms of action. In formula (XII), X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently CH or N. The base may be, for example, triethyl amine. The organic solvent may be, for example, 1,2-dichloroethane.

In some embodiments of the disclosure, the composition optionally comprises a carrier and/or a surfactant. In some embodiments of the disclosure, the composition further comprises at least one additional biologically-active compound. In some embodiments of the disclosure, the at least one additional biologically-active compound in the composition is a fungicide, an insecticide, a nematocide, a bactericide, a pharmaceutical, or an acaricide.

In an embodiment, the disclosure relates to a method of producing a novel DMOA proherbicide comprising condensation of a HPPD inhibitor with at least one fatty acid having 1-20 carbon atoms in the presence of a base (e.g., triethyl amine) and an organic solvent (e.g., 1,2-dichloroethane) in a single pot reaction. Suitable HPPD inhibitors include, but are not limited to, bicyclopyrone, fenquinotrione, nitisinone, 4-(2,6-diethyl-4-methylphenyl)-5-hydroxy-2,6-dimethyl-3(2H)-pyridazinone (PubChem CID 54683778), 4-(4-fluorophenyl)-6-[(2-hydroxy-6-oxo-1-cyclohexen-1-yl)carbonyl]-2-methyl-1,2,4-triazine-3,5(2H,4H)-dione (PubChem CID 71566698), 2-[hydroxy-[2-(3-methoxyphenyl)-1-(3-methoxypropyl)-6-oxopyrimidin-5-yl]methylidene]cyclohexane-1,3-dione (PubChem CID 90022616), and combinations thereof. In some embodiments, the disclosure relates to a proherbicide compound prepared by the aforementioned method.

In an embodiment, the disclosure relates to a method for controlling undesired vegetation, the method comprising contacting the vegetation or its environment with an effective amount of a proherbicide compound as taught herein, a derivative thereof, or a herbicidally-effective salt thereof.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1A, FIG. 1B, and FIG. 1C depict a schematic diagram of reactions to prepare novel DMOA proherbicides disclosed herein. FIG. 1A shows a schematic diagram of a reaction to prepare novel DMOA proherbicides of formula (IX) by condensing a HPPD inhibitor of formula (VIII) with a fatty acid or functionalized fatty acid. FIG. 1B shows a schematic diagram of reactions to prepare the endocyclic form and the exocyclic form of novel DMOA proherbicides of formula (XI) by respectively condensing the endocyclic enol form and the exocyclic enol form of a HPPD inhibitor of formula (X) with a fatty acid or functionalized fatty acid. FIG. 1C shows a schematic diagram of reactions to prepare the endocyclic form and the exocyclic form of novel DMOA proherbicides of formula (XIII) by respectively condensing the endocyclic enol form and the exocyclic enol form of a HPPD inhibitor of formula (XII) with a fatty acid or functionalized fatty acid.

FIG. 2A, FIG. 2B, and FIG. 2C depict images of a scan of 7-day-old lettuce (Lactuca sativa), bentgrass (Agrostis stolonifera), and Arabidopsis (Arabidopsis thaliana) seeds exposed to 1000 μM solution of the selected compounds. Seeds were germinating in 1000 μM solution for seven days. FIG. 2A, FIG. 2B, and FIG. 2C show pre-emergence activity results for AC-MA-07-02B (compound XIII-2, temborione-pelargonic acid conjugate), AC-MA-07-TT (the commercial triketone tembotrione), AC-MA-08-028 (compound XIII-3, sulcotrione-pelargonic acid conjugate), CN (water—negative control), AC-MA-08-ST (the commercial triketone sulcotrione), CS (10% acetone—solution control), AC-MA-06-MT (the commercial triketone mesotrione), and CP (the herbicide acifluorfen—positive control) for lettuce (three replicates in each row), bentgrass (three replicates in each row), and Arabidopsis (three replicates in each row) respectively.

FIG. 3 depicts an image of a scan of 31-day-old Arabidopsis (Arabidopsis thaliana) seedlings exposed when ten-days-old to 100 μM solution of the selected compounds. Ten-day-old seedlings were submerged in 100 μM solutions for 5 minutes and observed 21 days after treatment.

FIG. 4 depicts the electrolyte leakage (a measure of cellular damage) of 100 μM solutions of the compound (XIII-1), pelargonic acid, mesotrione, atrazine (positive control), and water (negative control).

FIG. 5A and FIG. 5B depict ¹H and ¹³C NMR spectra for the compound (XIII-1). FIG. 5A shows the ¹H NMR spectra, and FIG. 5B shows the ¹³C NMR spectra.

FIG. 6 depicts a schematic diagram of reactions to prepare certain keto-diesters disclosed herein. Formulas (I), (IV), and (VI) are carboxylic acids, formula (II) is a protected diester, formulas (III), (V), and (VII) are keto-diesters, where is n is independently 0, 1, 2, 3, 4, or 5; R₁ and R₂ are independently H or an alkyl group; X and Y are independently H, alkyl, NO₂, SO₂Me, CF₃, halide, NH, OH, SH or a derivative thereof, and Z is CH or N.

FIG. 7A and FIG. 7B depict images of a scan of 7-day-old Arabidopsis seedlings exposed to 1000 μM solution of the selected compounds. FIG. 7A shows results for water (negative control), 10% acetone (negative control), mesotrione (positive control), sulcotrione (positive control), and keto diesters of formula (III-1), (III-2), or (III-3). FIG. 7B shows results for 10% acetone (negative control), water (negative control), mesotrione (positive control), and keto diesters of formula (III-6), (VII-2), (III-5), (VI1), or (VII-1).

FIG. 8A and FIG. 8B depict ¹H and ¹³C NMR spectra for compound of formula (III-4).

FIG. 8A shows the ¹H NMR spectra, and FIG. 8B shows the ¹³C NMR spectra.

FIG. 9A and FIG. 9B depict ¹H and ¹³C NMR spectra for compound of formula (III-5).

FIG. 9A shows the ¹H NMR spectra, and FIG. 9B shows the ¹³C NMR spectra.

FIG. 10A and FIG. 10B depict ¹H and ¹³C NMR spectra for compound of formula (III-6). FIG. 10A shows the ¹H NMR spectra, and FIG. 10B shows the ¹³C NMR spectra.

FIG. 11A and FIG. 11B depict ¹H and ¹³C NMR spectra for compound of formula VII-1.

FIG. 11A shows the ¹H NMR spectra, and FIG. 11B shows the ¹³C NMR spectra.

FIG. 12A and FIG. 12B depict ¹H and ¹³C NMR spectra for compound of formula VII-2.

FIG. 12A shows the ¹H NMR spectra, and FIG. 12B shows the ¹³C NMR spectra.

### DETAILED DESCRIPTION

The inventors have discovered that compounds derived from a series of structurally-related triketone and keto diester herbicides that are 4-hydroxyphenyl-pyruvate dioxygenase (HPPD) inhibitors and linked to a fatty acid having 1 to 20 carbon atoms, such as the natural herbicide, pelargonic acid, and compositions comprising such compounds present with proherbicidal activity with two independent mechanisms of action. Disclosed herein are methods of preparing such compounds and compositions comprising such compounds. Also disclosed are methods for controlling undesired vegetation by contacting the undesired vegetation or its environment with an effective amount of such a compound or composition.

The present disclosure is based on the finding fatty acid (e.g., pelargonic acid) conjugates of a series of structurally related triketone and keto diester herbicides that are HPPD inhibitors (e.g., mesotrione) as defined herein, exhibit good proherbicidal activity with two independent mechanisms of action. Thus, according to the present disclosure there are provided compounds of formulas (IX), (XI), (XIII), or agronomically acceptable salts or zwitterionic species thereof for use as herbicides.

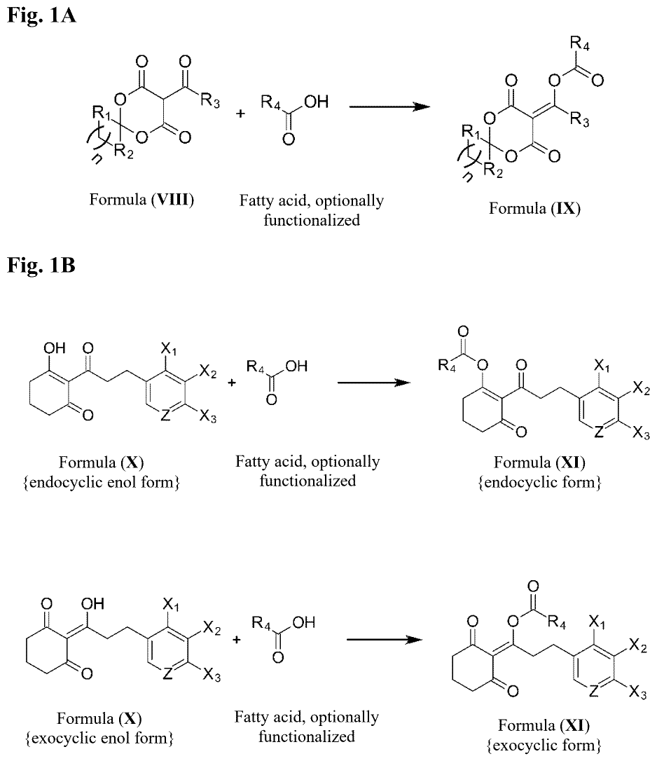

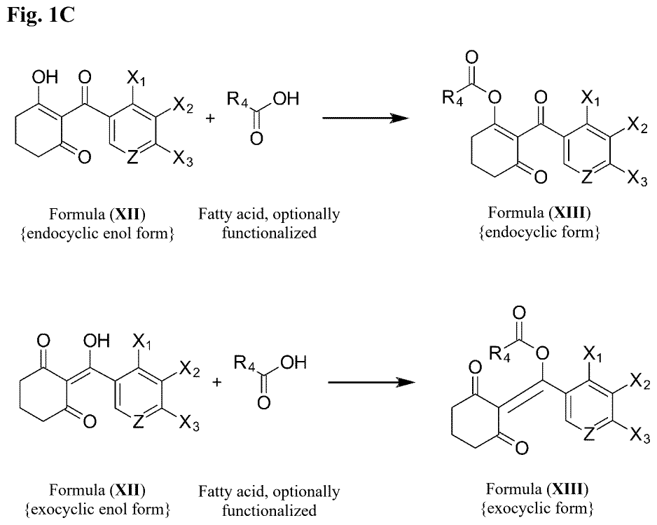

A schematic diagram of reactions to prepare novel DMOA proherbicides disclosed herein is depicted in FIG. 1A, FIG. 1B, and FIG. 1C. FIG. 1A shows a schematic diagram of a reaction to prepare novel DMOA proherbicides of formula (IX) by condensing a HPPD inhibitor of formula (VIII) with a fatty acid or functionalized fatty acid. FIG. 1B shows a schematic diagram of reactions to prepare the endocyclic form and the exocyclic form of novel DMOA proherbicides of formula (XI) by respectively condensing the endocyclic enol form and the exocyclic enol form of a HPPD inhibitor of formula (X) with a fatty acid or functionalized fatty acid. FIG. 1C shows a schematic diagram of reactions to prepare the endocyclic form and the exocyclic form of novel DMOA proherbicides of formula (XIII) by respectively condensing the endocyclic enol form and the exocyclic enol form of a HPPD inhibitor of formula (XII) with a fatty acid or functionalized fatty acid.

In accordance with an embodiment, the disclosure relates to a proherbicide compound having formula (IX)

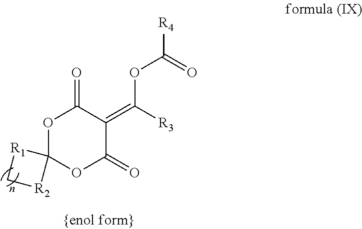

or a herbicidally-effective salt thereof,
wherein n is independently 0, 1, 2, 3, 4, or 5; R₁ and R₂ are independently H or an alkyl group;
R₄ is independently an alkyl chain having 1 to 20 carbon atoms, including, but not limited to alkyl chain with terminal hydroxy or halogen groups; and R₃ is independently

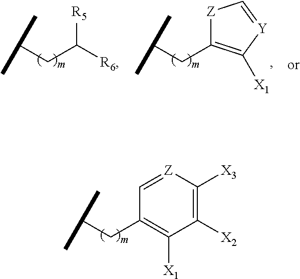

wherein m is independently 0, 1, 2, 3, 4, or 5; R₅ and R₆ are independently H, alkyl, aryl, heteroaryl, alkyl aryl, or alkyl heteroaryl; X₁, X₂, and X₃ are independently H, F, Cl, Br, I, NO₂, SCH₃, SO₂CH₃, or CH₂OR₇, wherein R₇ is independently any alkyl chain or halogenated alkyl chain; Y is independently NH, O, or S; and Z is independently CH or N.

In accordance with an embodiment, the disclosure relates to a method for preparing the proherbicide compound having formula (IX), above, or a herbicidally effective salt thereof, wherein the method comprises condensation of at least one tautomer of a HPPD inhibitor of formula (VIII)

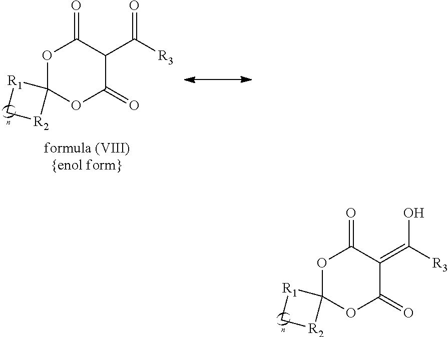

with at least one fatty acid having 6 to 20 carbon atoms including fatty acids with terminal functionalization in the presence of a base and an organic solvent in a single pot reaction. In formula (VIII), n is independently 0, 1, 2, 3, 4, or 5; R₁ and R₂ are independently H or an alkyl group; and R₃ is independently

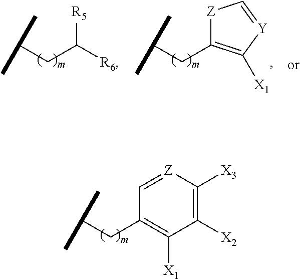

wherein m is independently 0, 1, 2, 3, 4, or 5; R₅ and R₆ are independently H, alkyl, aryl, heteroaryl, alkyl aryl, or alkyl heteroaryl; X₁, X₂, and X₃ are independently H, F, Cl, Br, I, NO₂, SCH₃, SO₂CH₃, or CH₂OR₇, wherein R₇ is independently any alkyl chain or halogenated alkyl chain; Y is independently NH, O, or S; and Z is independently CH or N. The base may be, for example, triethyl amine. The organic solvent may be, for example, 1,2-dichloroethane.

In accordance with an embodiment, the disclosure relates to a proherbicide compound having at least one tautomer of formula (XI)

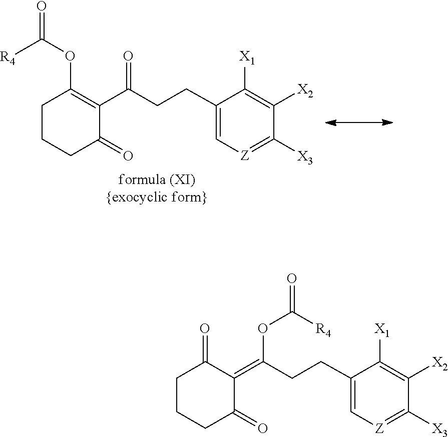

or a herbicidally-effective salt thereof;
wherein R₄ is independently an alkyl chain having 1 to 20 carbon atoms, including, but not limited to alkyl chain with terminal hydroxy or halogen groups; X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently CH or N.

In accordance with an embodiment, the disclosure relates to a method for preparing the proherbicide compound having formula (XI), above, or a herbicidally effective salt thereof, wherein the method comprises condensation of at least one tautomer of a HPPD inhibitor of formula (X)

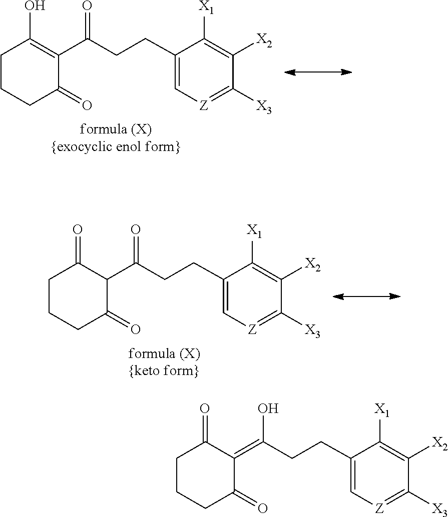

with at least one fatty acid having 6 to 20 carbon atoms including fatty acids with terminal functionalization in the presence of a base and an organic solvent in a single pot reaction. In formula (X), X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently CH or N. The base may be, for example, triethyl amine. The organic solvent may be, for example, 1,2-dichloroethane.

In accordance with an embodiment, the disclosure relates to a proherbicide compound having at least one tautomer of formula (XIII)

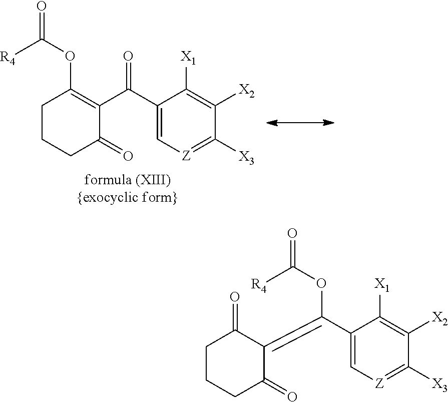

or a herbicidally-effective salt thereof,
wherein R₄ is independently an alkyl chain having 1 to 20 carbon atoms, including, but not limited to alkyl chain with hydroxy or halogen groups; X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently N; or
wherein R₄ is independently an alkyl chain having 7 to 20 carbon atoms, including, but not limited to alkyl chain with hydroxy or halogen groups; X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently CH or N; or
wherein R₄ is independently an alkyl chain having 1 to 20 carbon atoms, including, but not limited to alkyl chain with hydroxy or halogen groups; X₁, X₂, and X₃ are independently H, F, Cl, Br, I, NO₂, SCH₃, SO₂CH₃, or CH₂OR₇, wherein R₇ is independently any alkyl chain or functionalized alkyl chain, and wherein at least one of X₁, X₂, and X₃ is CH₂OR₇; and Z is independently CH or N.

In accordance with an embodiment, the disclosure relates to a method for preparing the proherbicide compound having formula (XIII), above, or a herbicidally effective salt thereof, wherein the method comprises condensation of at least one tautomer of a HPPD inhibitor of formula (XII)

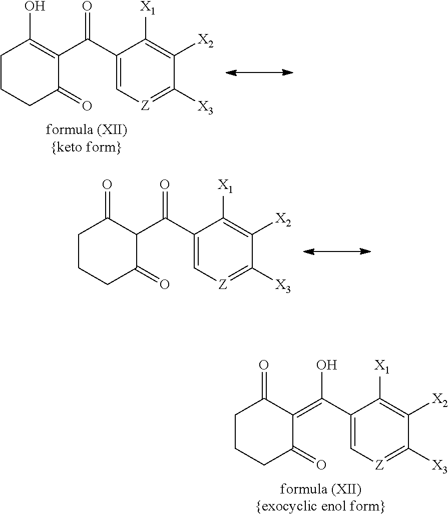

with at least one fatty acid or functionalized fatty acid having 6 to 20 carbon atoms in the presence of a base and a non-protic organic solvent in a single pot reaction to prepare a novel DMOA proherbicide with two independent mechanisms of action. In formula (XII), X₁, X₂, and X₃ are independently H, F, Cl, Br, I, NO₂, SCH₃, SO₂CH₃, or CH₂OR₇, wherein R₇ is independently any alkyl chain or functionalized alkyl chain; and Z is independently CH or N. The base may be, for example, triethyl amine. The organic solvent may be, for example, 1,2-dichloroethane.

Non-protic organic solvents include aprotic nonpolar organic solvents such as 1,2-dichloroethane, diethyl ether, toluene, and hexane, as well as aprotic polar organic solvents such as dimethyl sulfoxide (DMSO), acetonitrile, N,N-Dimethylformamide (DMF) and acetone.

In accordance with some embodiments of the disclosure, the composition optionally comprises a carrier and/or a surfactant. In some embodiments of the disclosure, the composition further comprises at least one additional biologically-active compound. In some embodiments of the disclosure, the at least one additional biologically-active compound in the composition is a fungicide, an insecticide, a nematocide, a bactericide, a pharmaceutical, or an acaricide.

In accordance with an embodiment, the disclosure relates to a method of producing a novel DMOA proherbicide comprising condensation of a HPPD inhibitor with at least one fatty acid having 1-20 carbon atoms in the presence of a base (e.g., triethyl amine) and an organic solvent (e.g., 1,2-dichloroethane) in a single pot reaction. Suitable HPPD inhibitors include, but are not limited to, bicyclopyrone, fenquinotrione, nitisinone, pogostone, 4-(2,6-diethyl-4-methylphenyl)-5-hydroxy-2,6-dimethyl-3(2H)-pyridazinone (PubChem CID 54683778), 4-(4-fluorophenyl)-6-[(2-hydroxy-6-oxo-1-cyclohexen-1-yl)carbonyl]-2-methyl-1,2,4-triazine-3,5(2H,4H)-dione (PubChem CID 71566698), 2-[hydroxy-[2-(3-methoxyphenyl)-1-(3-methoxypropyl)-6-oxopyrimidin-5-yl]methylidene]cyclohexane-1,3-dione (PubChem CID 90022616), and combinations thereof. In some embodiments, the disclosure relates to a proherbicide compound prepared by the aforementioned method.

In accordance with an embodiment, the disclosure relates to a method for controlling undesired vegetation, the method comprising contacting the vegetation or its environment with an effective amount of a proherbicide compound as taught herein, a derivative thereof, or a herbicidally-effective salt thereof.

“HPPD (4-hydroxyphenyl-pyruvate dioxygenase) inhibitors” are substances that inhibit the biosynthesis of 4-hydroxyphenyl-pyruvate dioxygenase. Examples of conventional HPPD inhibitors include: benzobicyclon; benzofenap; bicyclopyrone (4-hydroxy-3-[[2-[(2-methoxyethoxy)methyl]-6-(trifluoromethyl)-3-pyridinyl]carbonyl]bicyclo[3.2.1]oct-3-en-2-one); fenquinotrione (2-[[8-chloro-3,4-dihydro-4-(4-methoxyphenyl)-3-oxo-2-quinoxalinyl]carbonyl]-1,3-cyclohexanedione) {also referred to as 5-chloro-3-[(2-hydroxy-6-oxo-1-cyclohexen-1-yl)carbonyl]-1-(4-methoxyphenyl)-2-(1H)quinoxalinone)}; isoxachlortole; isoxaflutole; mesotrione; nitisinone (2-(2-nitro-4-(trifluoromethyl)benzoyl)cyclohexane-1,3-dione); pyrasulfotole; pyrazolynate; pyrazoxyfen; sulcotrione; tefuryltrione; tembotrione; topramezone; 4-(2,6-diethyl-4-methylphenyl)-5-hydroxy-2,6-dimethyl-3(2H)-pyridazinone (PubChem CID 54683778); 4-(4-fluorophenyl)-6-[(2-hydroxy-6-oxo-1-cyclohexen-1-yl)carbonyl]-2-methyl-1,2,4-triazine-3,5(2H,4H)-dione (PubChem CID 71566698); 5-[(2-hydroxy-6-oxo-1-cyclohexen-1-yl)carbonyl]-2-(3-methoxyphenyl)-3-(3-methoxypropyl)-4(3H)-pyrimidinone {also referred to as 2-[hydroxy-[2-(3-methoxyphenyl)-1-(3-methoxypropyl)-6-oxopyrimidin-5-yl]methylidene]cyclohexane-1,3-dione)}(PubChem CID 90022616); 2-methyl-N-(4-methyl-1,2,5-oxadiazol-3-yl)-3-(methylsulfinyl)-4-(trifluoromethyl) benzamide (PubChem CID 60194742); and 2-methyl-3-(methylsulfonyl)-N-(1-methyl-1H-tetrazol-5-yl)-4-(trifluoromethyl) benzamide (PubChem CID 60202789).

Of the conventional HPPD inhibitors listed above, the following HPPD inhibitors correspond to formula (XII) and can be converted to their corresponding proherbicides using the technology disclosed herein: mesotrione {Formula XII, X₁=NO₂, X₂=H, X₃=CO₂CH₃}; sulcotrione {Formula XII, X₁=Cl, X₂=H, X₃=SO₂}; tefuryltrione {Formula XII, X₁=Cl, X₂=CH₂OCH₂-2-furyl group, X₃=SO₂}; and tembotrione {Formula XII, X₁=Cl, X₂=CH₂OCH₂CF₃, X₃=SO₂}.

Other conventional HPPD inhibitors listed above (as well as additional HPPD inhibitors such as HPPD inhibitors corresponding to formula (X) and formula (VIII)) can also be converted to their corresponding proherbicides using the technology disclosed herein including, but not limited to, bicyclopyrone; fenquinotrione; nitisinone; topramezone; 4-(2,6-diethyl-4-methylphenyl)-5-hydroxy-2,6-dimethyl-3(2H)-pyridazinone (PubChem CID 54683778); 4-(4-fluorophenyl)-6-[(2-hydroxy-6-oxo-1-cyclohexen-1-yl)carbonyl]-2-methyl-1,2,4-triazine-3,5(2H,4H)-dione (PubChem CID 71566698); 5-[(2-hydroxy-6-oxo-1-cyclohexen-1-yl)carbonyl]-2-(3-methoxyphenyl)-3-(3-methoxypropyl)-4(3H)-pyrimidinone (also referred to as 2-[hydroxy-[2-(3-methoxyphenyl)-1-(3-methoxypropyl)-6-oxopyrimidin-5-yl]methylidene]cyclohexane-1,3-dione) (PubChem CID 90022616).

Examples of HPPD inhibitors corresponding to formula (X) that can be converted to their corresponding proherbicides using the technology disclosed herein include, but are not limited to, 2-(3-phenylpropanoyl)cyclohexane-1,3-dione (PubChem CID 44566543), 3-Hydroxy-2-(5′-phenylpent-4′E-enoyl)cyclohex-2-en-1-one (PubChem CID 90681779), 2-Cyclohexen-3-ol-1-one, 2-[11-phenylundecanoyl]-; 3-hydroxy-2-(11-phenylundecanoyl)cyclohex-2-en-1-one (PubChem CID 365014).

Examples of HPPD inhibitors corresponding to formula (VIII) that can be converted to their corresponding proherbicides using the technology disclosed herein include, but are not limited to, HPPD inhibitors of formula (III) such as 5-(2-chloro-4-(methylsulfonyl)benzoyl)-2,2-dimethyl-1,3-dioxane-4,6-dione (formula III-1), 2,2-dimethyl-5-(2-nitro-4-(trifluoromethyl)benzoyl)-1,3-dioxane-4,6-dione (formula III-2), 2,2-dimethyl-5-(4-(methylsulfonyl)-2-nitrobenzoyl)-1,3-dioxane-4,6-dione (formula III-3), 2,2-dimethyl-5-(3-phenylpropanoyl)-1,3-dioxane-4,6-dione (formula III-4), 8-(3-phenylpropanoyl)-6,10-dioxaspiro[4.5]decane-7,9-dione (formula III-5), and 5-(3-phenylpropanoyl)-2,2-dipropyl-1,3-dioxane-4,6-dione (formula III-6), set forth in the examples below; HPPD inhibitors of formula (V) such as 2,2-dimethyl-5-[2-(1-methylimidazol-4-yl)cyclopropanecarbonyl]-1,3-dioxane-4,6-dione (PubChem CID 126754540), 2-[(2-ethyl-4-methyl-1,3-benzoxazol-5-yl)-hydroxymethylidene]cyclohexane-1,3-dione (PubChem CID 21133763); and HPPD inhibitors of formula (VII) such as 2,2-dimethyl-5-(3-methylbutanoyl)-1,3-dioxane-4,6-dione (formula VII-1), and 8-(3-methylbutanoyl)-6,10-dioxaspiro[4.5]decane-7,9-dione (formula VII-2), set forth in the examples below.

U.S. Pat. No. 5,550,165 teaches pharmaceutical compositions capable of inhibiting the enzyme 4-hydroxyphenylpyruvate dioxygenase (HPPD) in mammals, and the preparation of pharmaceutical compositions for treating disorders and diseases in which intervention in the metabolic sequences catalyzed at least in part by HPPD is desirable. It is anticipated that at least some of the compounds presented in the current disclosure will also possess such properties. The compounds taught herein may also be useful as insecticides on blood-feeding insects (e.g., ticks, mosquitos, and bed bugs), as HPPD inhibitors have recently been found to be effective on these insects after a blood meal because the insects' HPPD is needed to detoxify high tyrosine levels from blood.

The term “fatty acid” as used herein is a carboxylic acid with an aliphatic chain, which is either saturated or unsaturated. In some embodiments, the fatty acid may have 6 to 20 carbon atoms. In some embodiments, the fatty acid may be functionalized including, but not limited to, terminal functionalization such as terminal hydroxy or halogen groups. Examples of suitable fatty acids include, but are not limited to, caproic acid, heptanoic acid, octanoic acid, nonanoic acid, capric acid, undecanoic acid, lauric acid, tridecanoic acid, tetradecanoic acid, pentadecanoic acid, palmitic acid, heptadecanoic acid, stearic acid, palmitoleic acid, oleic acid, linoleic acid, linolenic acid, and gamma-linolenic acid, each of which may optionally be functionalized. For example, as used herein, the term “fatty acid” includes nonanoyl chloride, which is nonanoic acid that is terminally functionalized with a chlorine atom.

As used herein, the term, “alkyl” means a straight-chain, branched open-chain, or branched cyclic-chain saturated hydrocarbon group which is optionally mono- or polysubstituted, and in the latter case is referred to as “substituted alkyl”. Substituents may be halogen atoms, alkoxy, haloalkoxy, cyano, alkylthio, haloalkylthio, amino or nitro groups. In some embodiments the substituents are methoxy, methyl, fluoroalkyl, cyano, nitro, fluorine, chlorine, bromine, or iodine. The prefix “bis” also includes identical alkyl groups, e.g., dimethyl, diethyl, dipropyl, a combination of different alkyl groups, e.g., methyl(ethyl) or ethyl(methyl), or cyclic groups, e.g., —CH₂(CH₂)ₙCH₂— wherein n=2, 3, or 4. The term aryl includes both substituted and unsubstituted phenyl, pyridyl, naphthyl, anthracenyl, imidazolyl, oxazolyl, thiazolyl.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic.

The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

New herbicide classes are needed to increase the number of bio-based herbicides available, and to overcome the rapid evolution of target-site and non-target-site-based herbicide resistance in weeds. The compounds described herein may be used for both weed management and herbicide resistance management.

It may be beneficial to apply a DMOA proherbicide of the disclosure alone or in combination with other herbicides, or in a mixture with other crop protection agents, for example together with agents for controlling arthropod pests, phytopathogenic fungi, or bacteria. Also of interest is the miscibility a DMOA proherbicide of the disclosure with mineral salt solutions, which are employed for treating nutritional and trace element deficiencies. Other additives such as non-phytotoxic oils and oil concentrates may also be added.

As used herein, the term “effective amount” denotes an amount of a DMOA proherbicide of the disclosure, which is sufficient for controlling undesired vegetation. Such an amount can vary in a broad range and is dependent on various factors, such as the undesired vegetation species or biotype to be controlled, the developmental stage of the target weed, the climatic conditions, and the specific DMOA proherbicide of the disclosure used.

The DMOA proherbicides of the disclosure may be converted into customary types of compositions such as solutions, emulsions, suspensions, dusts, powders, pastes, granules, pressings, capsules, or mixtures thereof. Examples for composition types are suspensions, emulsifiable concentrates, emulsions, capsules, pastes, pastilles, wettable powders, wettable dusts, pressings, granules, insecticidal articles, or gel formulations. These and further formulation types are defined in the “Catalogue of pesticide formulation types and international coding system”, Technical Monograph No. 2, 6.sup.th Ed. May 2008, CropLife International.

A composition of the disclosure may comprise a DMOA proherbicide of the disclosure and optionally a carrier. Examples of suitable carriers are solvents, liquid carriers, solid carriers, fillers, surfactants, dispersants, emulsifiers, wetting agents, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers, and binders.

A solvent may be water or an organic solvent. An organic solvent may be a mineral oil fraction, a vegetable oil, an oil of animal origin, an aliphatic hydrocarbon, a cyclic hydrocarbon, an aromatic hydrocarbon, an alcohol, dimethyl sulfoxide (DMSO), a ketone, a glycol, an ester, a fatty acid, a phosphonate, an amine, an amide, or a mixture thereof. A hydrocarbon may be toluene, paraffin, tetrahydronaphthalene, or alkylated naphthalenes. An alcohol may be at least one of ethanol, propanol, butanol, benzylalcohol, or cyclohexanol. A ketone may be at least one of acetone, pentanone, or cyclohexanone. An ester may be at least one of lactate, carbonate, fatty acid ester, or gamma-butyrolactone. Non-protic organic solvents include aprotic nonpolar organic solvents such as 1,2-dichloroethane, diethyl ether, toluene, and hexane, as well as aprotic polar organic solvents such as dimethyl sulfoxide (DMSO), acetonitrile, N,N-dimethylformamide (DMF) and acetone

A composition disclosed herein may be used with at least one solid carrier or filler such as a mineral earths, fertilizer, product of vegetable origin, and mixtures thereof. A mineral earth may be, for example, silicate, silica gel, talc, kaolin, limestone, lime, chalk, clay, dolomite, diatomaceous earth, bentonite, calcium sulfate, magnesium sulfate, or magnesium oxide. A fertilizer may be, for example, ammonium sulfate, ammonium phosphate, ammonium nitrate, or urea. A product of vegetable origin may be starch, cellulose, cereal meal, tree bark meal, wood meal, or nutshell meal.

Suitable surfactants for use in a composition disclosed herein may be, for example, at least one surface-active compound, block polymer, polyelectrolyte, or mixtures thereof. A surface-active compound may be, for example, an anionic, cationic, nonionic, or amphoteric surfactant. Examples of surfactants are listed in McCutcheon's, Vol. 1: Emulsifiers & Detergents, McCutcheon's Directories, Glen Rock, USA, 2020 (International Ed. or North American Ed.). Such surfactants can be used at least as emulsifier, dispersant, solubilizer, wetter, penetration enhancer, protective colloid, or adjuvant.

Suitable anionic surfactants may be alkali, alkaline earth, ammonium salts of sulfonates, sulfates, phosphates, carboxylates, and mixtures thereof. Examples of sulfonates are alkylarylsulfonates, diphenylsulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants may be alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants may be quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants may be compounds which have a neglectable or even no pesticidal activity themselves and which improve the biological performance of the DMOA proherbicides of formula (III) on the target. Examples are surfactants and mineral or vegetable oils. Suitable thickeners are polysaccharides (such as xanthan gum, carboxymethylcellulose), inorganic clays (organically modified or unmodified), polycarboxylates, and silicates. Suitable bactericides are bronopol and isothiazolinone derivatives such as alkylisothiazolinones and benzisothiazolinones. Suitable anti-freezing agents are ethylene glycol, propylene glycol, urea and glycerin. Suitable anti-foaming agents are silicones, long chain alcohols, and salts of fatty acids.

Suitable colorants (such as red, blue, or green) are pigments of low water solubility and water-soluble dyes. Examples are inorganic colorants (such as iron oxide, titan oxide, iron hexacyanoferrate) and organic colorants (such as alizarin-, azo- and phthalocyanine colorants). Suitable tackifiers or binders are polyvinylpyrrolidons, polyvinylacetates, polyvinyl alcohols, polyacrylates, biological or synthetic waxes, and cellulose ethers.

The inventors have discovered that certain keto-diester compounds derived from carboxylic acids and compositions comprising such compounds present with herbicidal activity. Disclosed herein are methods of preparing such compounds and compositions comprising such compounds. Also disclosed are methods for controlling undesired vegetation by contacting the undesired vegetation or its environment with an effective amount of such a compound or composition. In an embodiment of the present invention, the aforementioned keto-diester compounds (which are the subject of the inventors' prior patent application, U.S. patent application Ser. No. 18/775,040, filed Jul. 17, 2024) may be utilized as a HPPD inhibitor reactant (i.e., formula (VIII) in reaction shown in FIG. 1A).

Aspects of the present disclosure are based on the finding that certain keto-diester derivatives as defined herein, exhibit good herbicidal activity. Thus, according to the present disclosure there is provided a compound of formula (VIII) or an agronomically acceptable salt or zwitterionic species thereof for use as a herbicide.

Aspects of the disclosure relate to keto-diesters of formula (VIII):

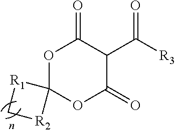

or a herbicidally-effective salt thereof, where n is independently 0, 1, 2, 3, 4, or 5; R₁ and R₂ are independently H or an alkyl group; R₃ is independently

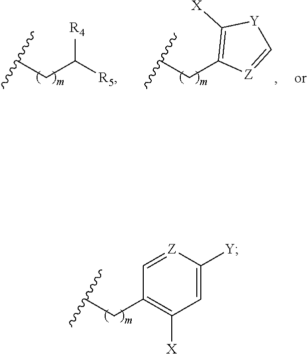

where m is independently 0, 1, 2, 3, 4, 5; R₄ and R₅ are independently H, alkyl, aryl, heteroaryl, alkyl aryl, alkyl heteroaryl; X and Y are independently H, alkyl, NO₂, SO₂Me, CF₃, halide, NH, OH, SH or a derivative thereof, and Z is independently CH or N.

In an embodiment, the disclosure relates to a method for preparing certain keto-diesters or herbicidally-effective salts thereof. The method comprises condensation of a protected diester of formula (II)

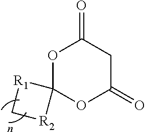

with at least one alkyl or aryl acid in the presence of an activating agent and a base in a single pot reaction to prepare certain keto-diesters. In formula II, n is independently 0, 1, 2, 3, 4, or 5; and R₁ and R₂ are independently H or an alkyl group. The readily available alkyl, aryl, heteroaryl, alkyl aryl, or alkyl heteroaryl acids may be activated in situ with an activating agent prior to condensation in a one pot reaction to yield certain keto-diesters. The activating agent may be a diimide, an inorganic chloride, or sterically hindered aromatic acid anhydride. The base may be a basic resin such as AMBERLITE IRA-410, DOWEX 1-X8, or AMBERCHROM 1X4 chloride form.

AMBERLITE IRA-410 chloride form is a strongly basic anion exchange resin. AMBERLITE is a registered trademark of DDP Specialty Electronic Materials; as a material for use in removing or replacing metallic or non-metallic ions or acid-forming Constituents from Fluids. DOWEX 1-X8 chloride form is a strongly basic anion exchanger. DOWEX is a trademark of The Dow Chemical Company for ionic and cationic synthetic resins.

AMBERCHROM 1X4 chloride form is a fine mesh anion exchange resin. AMBERCHROM is a registered trademark of DDP Specialty Electronic Materials; as chromatographic resins and columns for use in separation, filtration, and purification of liquids and solids.

The at least one substituted alkyl, aryl acid may be 4-methylsulfonyl-2-nitrobenzoic acid, 2-chloro-4-(methylsulfonyl) benzoic acid, isovaleric acid, or phenyl propanoic acid. The base may be dimethylamino pyridine, pyridine, N-methyl morpholine.

The method for preparing the compounds taught herein may comprise adding at room temperature N, N′-dicyclohexylcarbodiimide (DCC) to a solution of a carboxylic acid in anhydrous dimethyl formamide (DMF). After cooling to 0° C., adding the protected diester, followed by N, N′-dimethylamino pyridine (DMAP). The reaction mixture may then be heated to 100° C. and stirred at the same temperature for about 30 to 45 hours. The method further comprises washing and drying the product, followed by purification. The simple synthesis of these compounds may provide a cost advantage over many currently-used commercial products that could be passed on to the farmer.

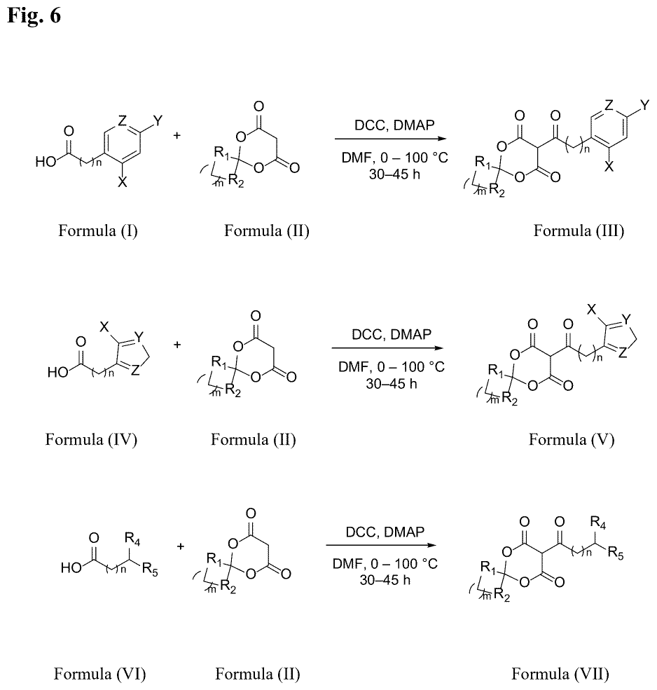

As seen in the schematic diagram of FIG. 6, the reactant keto-diesters of formula (VIII) in the schematic diagram of FIG. 1A may be prepared by reacting a compound of formula (I) with a compound of formula (II) to obtain a compound of formula (III)

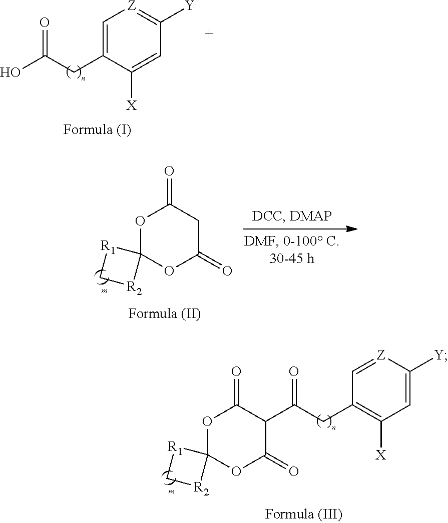

or reacting a compound of formula (IV) with a compound of formula (II) to obtain a compound of formula (V)

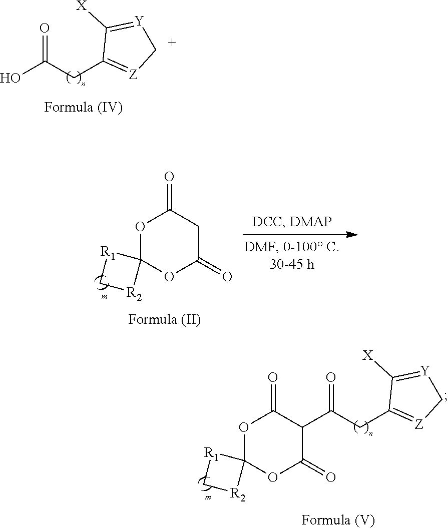

reacting a compound of formula (VI) with a compound of formula (II) to obtain a compound of formula (VII)

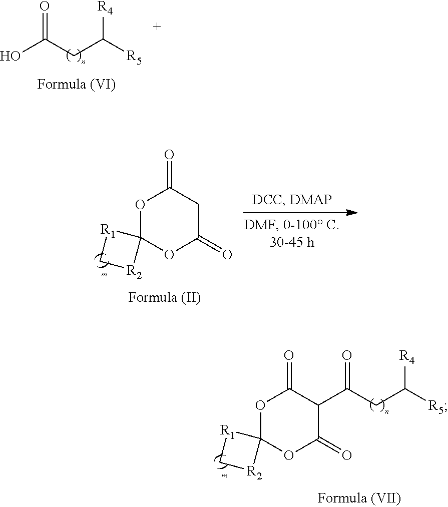

where is n is independently 0, 1, 2, 3, 4, or 5; R₁ and R₂ are independently H or an alkyl group; X and Y are independently H, alkyl, NO₂, SO₂Me, CF₃, halide, NH, OH, SH or a derivative thereof, and Z is CH or N.

In embodiments, the DMOA proherbicide molecules disclosed herein are designed to break down into a fatty acid and a HPPD inhibitor once systemically absorbed by the plant. For example, the DMOA proherbicide molecules of formula (IX) are designed to break down into a fatty acid (e.g., pelargonic acid) and a keto-diester of formula (VIII) or a herbicidally-effective salt thereof once systemically absorbed by the plant. Application of a keto-diester of formula (VIII) or a herbicidally-effective salt thereof as one of the dual breakdown products of the DMOA proherbicide of formula (IX); where n is 0, 1, 2, 3, 4, or 5; R₁ and R₂ are independently H or an alkyl group; R₃ is independently

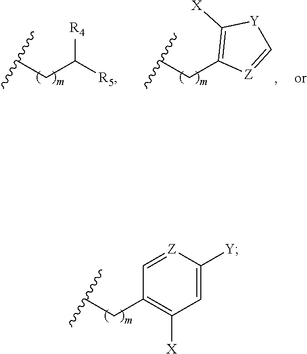

where m is independently 0, 1, 2, 3, 4, 5; R₄ and R₅ are independently H, alkyl, aryl, heteroaryl, alkyl aryl, alkyl heteroaryl; X and Y are independently H, alkyl, NO₂, SO₂Me, CF₃, halide, NH, OH, SH or a derivative thereof, and Z is independently CH or N, or a derivative thereof according to the disclosure, or compositions comprising them, may be performed by applying from a pre-dosage device, a knapsack sprayer, a spray tank, a spray plane, or an irrigation system. The composition may comprise water, buffer, and/or further adjuvants or carriers to the desired application concentration and the ready-to-use spray liquor or the composition according to the disclosure is thus obtained. Usually, 20 to 2000 liters, preferably 50 to 400 liters, of the ready-to-use spray liquid are applied per hectare of agricultural useful area.

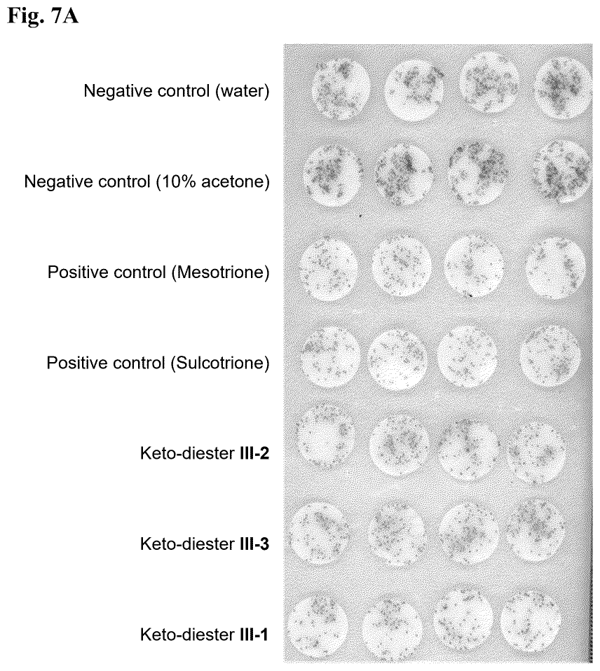

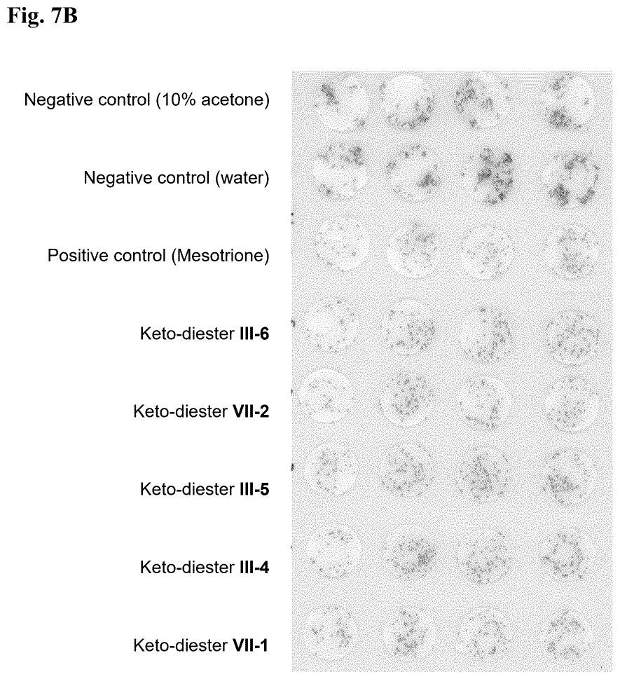

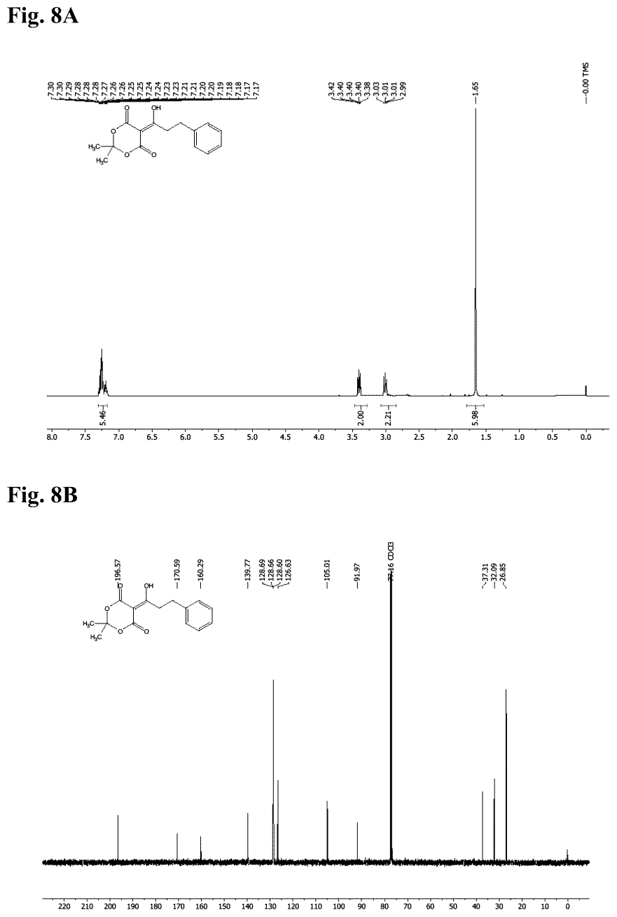

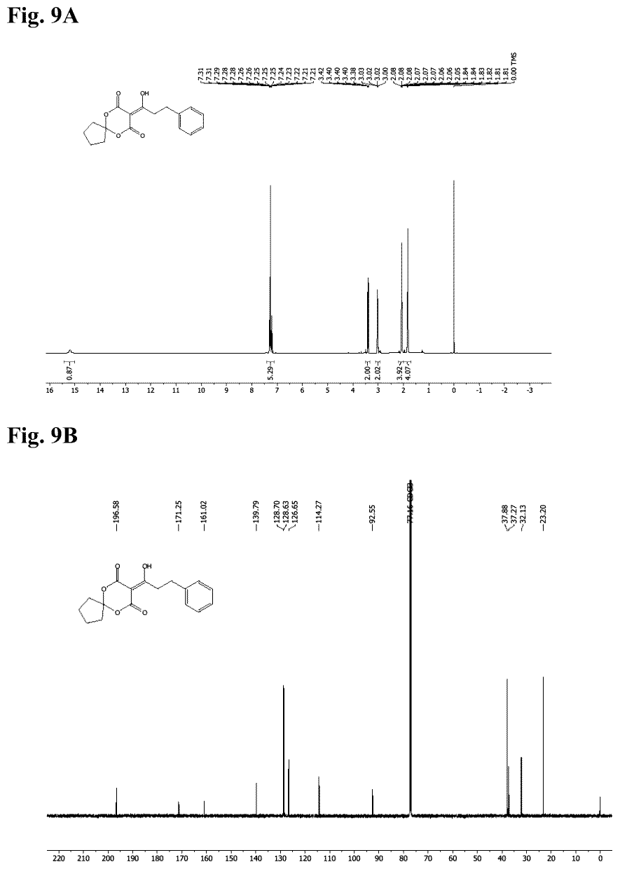

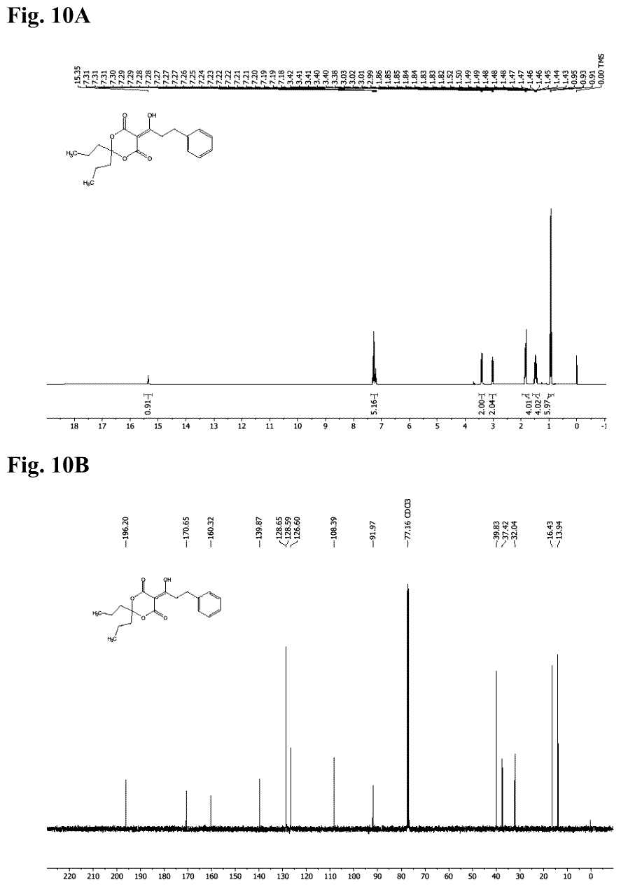

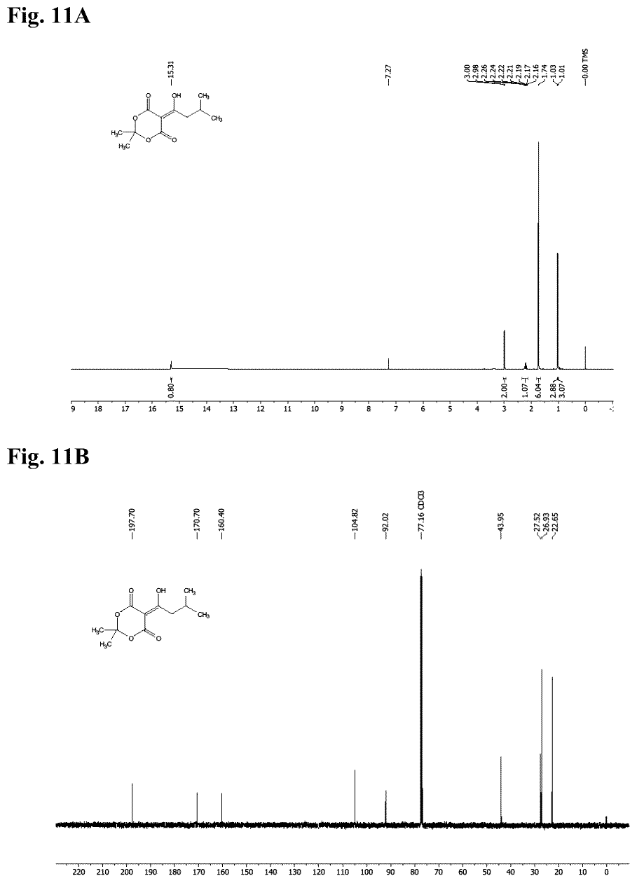

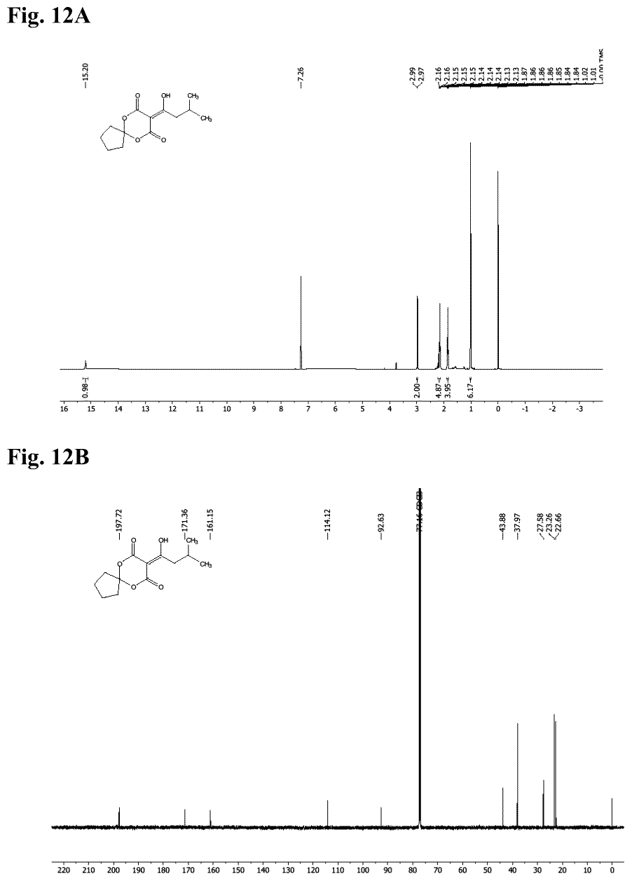

The phytotoxic activity of compounds belonging to formula (III), (V), or (VII) classes were evaluated in a primary bioassay. The synthetic triketone herbicides mesotrione and sulcotrione were used as positive controls. The seeds of monocot bentgrass (Agrostis stolonifera) and dicot Arabidopsis (Arabidopsis thaliana) were exposed for 7 days to 1000 μM solution of each chemical. For example, within the III series, the compound III-2 significantly reduced growth of Agrostis seedlings: most of them developed symptoms of chlorosis, some pale green and with developed, shorter leaves compared to negative controls containing H₂O or 10% acetone. The seedlings growing with compounds III-3 and III-1 did not develop chlorosis although growth of some of them was reduced. Arabidopsis seedling growth was significantly affected by all of the tested compounds. As seen in FIG. 7A and FIG. 7B, almost all 7-days-old seedlings exposed to any of the new compounds were chlorotic. Chlorosis of treated seedlings is a common symptom of HPPD enzyme inhibition, and the obtained results are an initial confirmation this mode of action for these new compounds. Next, a detailed assessment of phytotoxic activity of potential herbicide candidates was accomplished by measurement a growth of duckweed (Lemna paucicostata) frond surface area (Table 2). Mesotrione and sulcotrione were used as positive controls. In FIG. 7A, the compound of formula III-2 was the most phytotoxic compound tested, with an IC50 of 1.9 μM, while the remaining two chemicals tested displayed reduced phytotoxic activity with an IC50 of 46 μM for compound (III-1), and 192.9 M for compound III-3.

The most active of these compounds (III-2) is more active than the commercial herbicides bentazon, clomazone, glyphosate, glufosinate, asulam, chloropropham, and S-ethyl dipropyl thiocarbamate (EPTC) in the same bioassay (see Michel, A., et al. (2004) “Dose-response relationships between herbicides with different modes of action and growth of Lemna paucicostata—an improved ecotoxicological method,” Environ. Toxicol. Chem. 23(4): 1074-1079).

The keto-diesters of formula (VIII) are suitable as herbicides. They may be used as one of the dual breakdown products of the DMOA proherbicides of formula (IX), herbicides individually, as a mixture, or as an appropriate composition.

Keto-diesters of formula (VIII) may be employed as breakdown products within plants mainly by spraying the leaves with the DMOA proherbicides of formula (IX) or compositions comprising them. Here, the application can be carried out using, for example, water as carrier and customary spraying techniques using spray liquor amounts of from about 50 to 1000 L/ha (for example from 300 to 400 L/ha). The DMOA proherbicides disclosed herein, or compositions comprising them may also be applied by the low-volume or the ultra-low-volume method, or in the form of microgranules.

A DMOA proherbicide disclosed herein or a composition comprising such may be applied before, during, and/or after emergence of the undesired vegetation. Similarly, a DMOA proherbicide disclosed herein or a composition comprising such may be applied before or during sowing.

The presence of one or more possible asymmetric carbon atoms in a compound of formula (VIII) means that the compound may occur in chiral isomeric form, i.e., enantiomeric or diastereomeric form. Atropisomers may also occur as a result of restricted rotation about a single bond. Formula (VIII) is intended to include all those possible isomeric forms and mixtures thereof. The present disclosure includes all those possible isomeric forms and mixtures thereof for a keto-diester of formula (VIII). Likewise, keto-diesters of formula (VIII) are intended to include all possible tautomers (including lactam-lactim tautomerism and keto-enol tautomerism) where present. The present disclosure includes all possible tautomeric forms for a compound of formula (VIII). Similarly, where there are di-substituted alkenes, these may be present in E or Z form or as mixtures of both in any proportion. The present disclosure includes all these possible isomeric forms and mixtures thereof for a compound of formula (VIII).

A DMOA proherbicide disclosed herein will typically be provided in the form of an agronomically acceptable salt, a zwitterion, or an agronomically acceptable salt of a zwitterion. This disclosure covers all such agronomically acceptable salts, zwitterions, and mixtures thereof in all proportions.

All compounds of formula (VIII) can be present as equilibrium of tautomers. For example, ketones with an α-hydrogen can exist in an equilibrium of the keto form and the enol form.

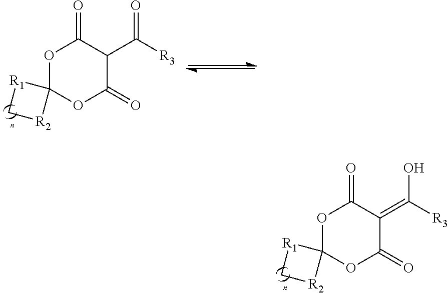

Unless stated to the contrary, the disclosure includes all such possible tautomers.

Suitable agronomically acceptable salts useful in the present disclosure can be with cations that include but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium, and tripropylsulfoxonium.

Compounds disclosed herein can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the disclosure with other herbicides can broaden the spectrum of activity against additional weed species, and suppress the proliferation of any resistant biotypes. Thus, the present disclosure also pertains to a composition comprising a compound as described (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures comprising the DMOA proherbicides taught herein, one or more other biologically active compounds or agents can be formulated together with a compound of as described to form a premix, or one or more other biologically active compounds or agents can be formulated separately from the compound as claimed, and the compositions combined together before application (e.g., in a spray tank) or alternatively applied in succession.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

As used herein, the term “about” is defined as plus or minus ten percent of a recited value. For example, about 1.0 g means 0.9 g to 1.1 g.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of excludes any element, step, or ingredient not specified. If in the claim, such would close the claim to the inclusion of materials other than those recited except for impurities ordinarily associated therewith. When the phrase” consisting of appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed composition. The term” consisting essentially of occupies a middle ground between “comprising” and “consisting of”.

As referred to herein, the term “seedling”, used either alone or in a combination of words means a young plant developing from the embryo of a seed.

Compounds described herein can exist as one or more stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects stereoisomer(s). Additionally, the skilled artisan knows how when enriched relative to the other stereoisomer(s) or when separated from the other to separate, enrich, and/or to selectively prepare said stereoisomers. The compounds described herein may be present as a mixture of stereoisomers, individual stereoisomers, as an optically active form, or as chiral salts of racemic stereoisomers.

Embodiments of the present disclosure are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1

### Synthesis of DMOA Proherbicides

A general synthetic route for the preparation of DMOA proherbicides is shown below. The newly prepared DMOA proherbicides have formula (XIII) {endocyclic form} and/or formula (XIII) {exocyclic form}, depending on the equilibrium state of the endocyclic enol and exocyclic enol conformations of the reactant HPPD of formula (XII). Typically, the endocyclic enol form of the reactant HPPD of formula (XII) is dominant in aqueous solutions and, as a consequence, the endocyclic form of the newly prepared DMOA proherbicides having formula (XIII) will be dominant as well. In formula (XII) and formula (XIII), R₄ is independently an alkyl chain having 1 to 20 carbon atoms, including, but not limited to alkyl chain with terminal hydroxy or halogen groups; X₁, X₂, and X₃ are independently H, F, Cl, Br, I, NO₂, SCH₃, SO₂CH₃, or CH₂OR₇, where R₇ is independently any alkyl chain or functionalized alkyl chain; and Z is independently CH or N.

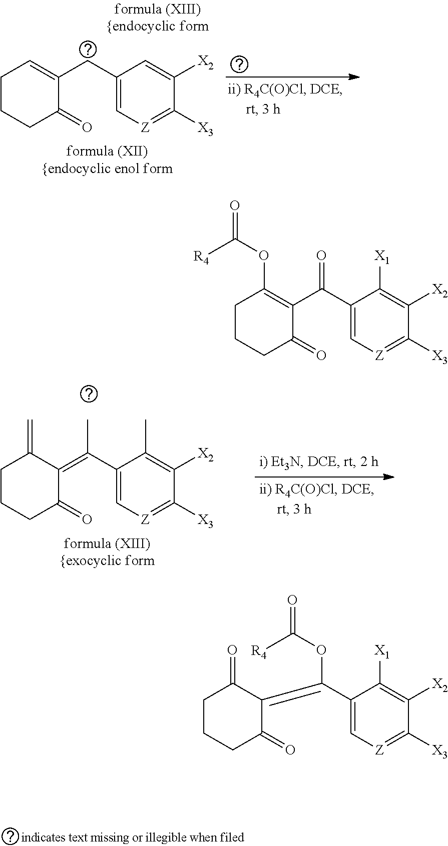

An exemplary DMOA proherbicide having formula (XIII) {endocyclic form} was prepared by adding triethyl amine (Et₃N) (20 mL) to a solution of mesotrione (4.5 g, 13.26 mmol) (i.e., mesotrione, shown below, is an HPPD inhibitor of formula (XII) {endocyclic enol form is dominant in this solution}) in 1,2-dichloroethane (DCE) (40 mL) and stirred for 2 hr at room temperature. The solvent was then removed in a rotary evaporator to produce bright-yellow-colored crystals. Without further purification, these crystals were resuspended in 1,2-dichloroethane (DCE) (40 mL) and nonanoyl chloride (2.34 g, 13.26 mmol) was added at room temperature. After 3 h of stirring, the solvent was evaporated and purified by flash-column chromatography on silica to produce the yellow colored liquid pelargonic acid conjugate of mesotrione. Yield was 48%. The synthetic route for the preparation of the exemplary DMOA proherbicide, 2-(4-(methylsulfonyl)-2-nitrobenzoyl)-3-oxocyclohex-1-en-1-yl nonanoate (compound (XIII-1)), is shown below.

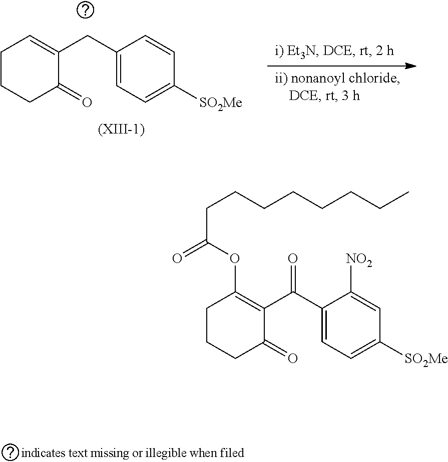 [SMILES: CS(=O)(=O)c1ccc(CC2=CCCCC2=O)cc1 ; CCCCCCCCC(=O)OC1=C(C(=O)c2ccc(S(C)(=O)=O)cc2[N+](=O)[O-])C(=O)CCC1]

Other DMOA proherbicides of formula (XIII) may be produced by this same general synthetic route. For example, compound (XIII-2) (temborione-pelargonic acid conjugate, also referred to herein as AC-MA-07-02B) and compound (XIII-3) (sulcotrione-pelargonic acid conjugate, also referred to herein as AC-MA-08-028) may be produced by this same general synthetic route, as shown below. Likewise, DMOA proherbicides of formula (XI) and (IX) may be produced by this same general synthetic route.

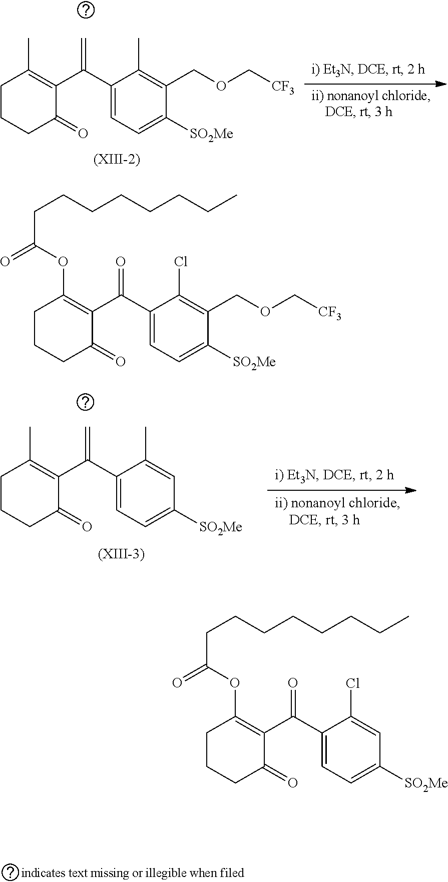

The reactant IPPD inhibitors of formula (X) and formula (XII) exist in different tautomeric forms according to the environmental conditions in which they are found, such as solution pH. By the controlling environment conditions, particularly solution pH, it may be possible to drive the reaction product (i.e., the DMOA proherbicides of formula (XI) and formula (XIII), respectively) toward/away from one tautomeric form relative to the other (endocyclic form vs exocyclic form).

Exemplary DMOA proherbicides prepared by this method were 2-(4-(methylsulfonyl)-2-nitrobenzoyl)-3-oxocyclohex-1-en-1-yl nonanoate (compound (XIII-1)), temborione-pelargonic acid conjugate (compound (XIII-2)), and sulcotrione-pelargonic acid conjugate (compound (XIII-3)).

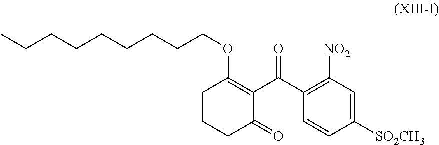 [SMILES: CCCCCCCCCOC1=C(C(=O)c2ccc(S(C)(=O)=O)cc2[N+](=O)[O-])C(=O)CCC1]

XIII-1=2-(4-(methylsulfonyl)-2-nitrobenzoyl)-3-oxocyclohex-1-en-1-yl nonanoate (R₄=CH₃(CH₂)₇—, X₁=NO2, X₂=H, X₃=SO₂CH₃, and Z=CH), ¹H NMR (400 MHz, CDCl₃) δ 8.61 (d, J=1.7 Hz, 1H), 8.20 (dd, J=8.0, 1.7 Hz, 1H), 7.60 (d, J=7.9 Hz, 1H), 3.13 (s, 3H), 2.77 (t, J=6.2 Hz, 2H), 2.54 (t, J=7.6 Hz, 2H), 2.44 (dd, J=7.6, 5.9 Hz, 2H), 2.08 (p, J=6.4 Hz, 2H), 1.68 (p, J=7.5 Hz, 2H), 1.39-1.20 (m, 11H), 0.91-0.82 (m, 3H). ¹³C NMR (101 MHz, CDCl₃) δ 196.1, 188.1, 175.8, 169.9, 146.2, 142.4, 142.3, 132.4, 129.3, 125.2, 123.5, 44.5, 37.4, 34.4, 31.9, 31.2, 29.2, 29.2, 29.1, 24.3, 22.7, 19.6, 14.2. HRMS (ESI) m/z calculated for C₂₃H₂₈NO₈S [M-H]⁻, 478.1541, found 478.1538.

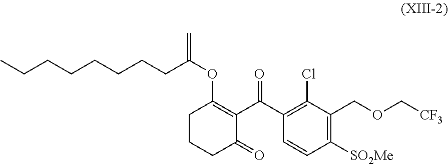 [SMILES: C=C(CCCCCCCC)OC1=C(C(=O)c2ccc(S(C)(=O)=O)c(COCC(F)(F)F)c2Cl)C(=O)CCC1]

XIII-2=Tembotrione-pelargonic acid conjugate (R₄=CH₃(CH₂)₇—, X₁=Cl, X₂=CH₂OCH₂CF₃, X₃=SO₂CH₃, and Z=CH), ¹H NMR (500 MHz, CDCl3) δ 8.11 (d, J=8.2 Hz, 1H), 7.65 (d, J=8.2 Hz, 1H), 5.34 (s, 2H), 4.01 (q, J=8.6 Hz, 2H), 3.20 (s, 3H), 2.75 (t, J=6.2 Hz, 2H), 2.53-2.45 (m, 2H), 2.39 (t, J=7.5 Hz, 2H), 2.16-2.08 (m, 2H), 1.60 (p, J=7.5 Hz, 2H), 1.35-1.18 (m, 10H), 0.86 (t, J=6.9 Hz, 3H). ¹³C NMR (126 MHz, CDCl3) δ 196.1, 190.4, 172.1, 170.0, 144.4, 143.3, 134.8, 134.0, 130.1, 128.7, 128.4, 68.6 (q, J=34.4 Hz), 66.2, 45.6, 37.2, 34.2, 31.8, 30.1, 29.2, 29.1, 29.0, 24.4, 22.7, 20.2, 14.2. HRMS (ESI) m/z calculated for C₂₆H₃₁ClF₃O₇S [M-H]⁻, 579.1473, found 579.1465.

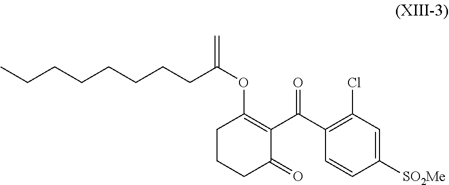 [SMILES: C=C(CCCCCCCC)OC1=C(C(=O)c2ccc(S(C)(=O)=O)cc2Cl)C(=O)CCC1]

XIII-3=sulcotrione-pelargonic acid conjugate (R₄=CH₃(CH₂)₇—, X₁=Cl, X₂=H, X₃—SO₂CH₃, and Z=CH), ¹H NMR (400 MHz, CDCl₃) δ 7.93 (d, J=1.7 Hz, 1H), 7.85 (dd, J=8.1, 1.7 Hz, 1H), 7.74 (d, J=8.1 Hz, 1H), 3.06 (s, 3H), 2.74 (t, J=6.2 Hz, 2H), 2.51 (dd, J=7.5, 5.9 Hz, 2H), 2.37 (t, J=7.5 Hz, 2H), 2.17-2.06 (m, 2H), 1.58 (p, J=7.4 Hz, 2H), 1.34-1.19 (m, 10H), 0.88-0.81 (m, 3H). ¹³C NMR (101 MHz, CDCl₃) δ 196.23, 190.1, 171.3, 170.0 143.5, 142.4, 133.0, 131.3, 129.5, 128.6, 125.8, 44.4, 37.1, 34.2, 31.8, 29.9, 29.2, 29.1, 29.0, 24.4, 22.7, 20.2, 14.2. HRMS (ESI) m/z calculated for C₂₃H₂₈ClO₆S [M-H]⁻, 467.1301, found 467.1305.

The data presented here shows that DMOA proherbicides of formula (XIII), where R₄ is independently an alkyl chain having 1 to 20 carbon atoms, including, but not limited to alkyl chain with terminal hydroxy or halogen groups; X₁, X₂, and X₃ are independently H, F, Cl, Br, I, NO₂, SCH₃, SO₂CH₃, or CH₂OR₇, where R₇ is independently any alkyl chain or functionalized alkyl chain; and Z is independently CH or N may be prepared in a one to two step reaction.

### Example 2

### Phytotoxic Activity of DMOA Proherbicides

The effects of the DMOA proherbicides of the disclosure in the growth of monocot and dicot seedlings were tested.

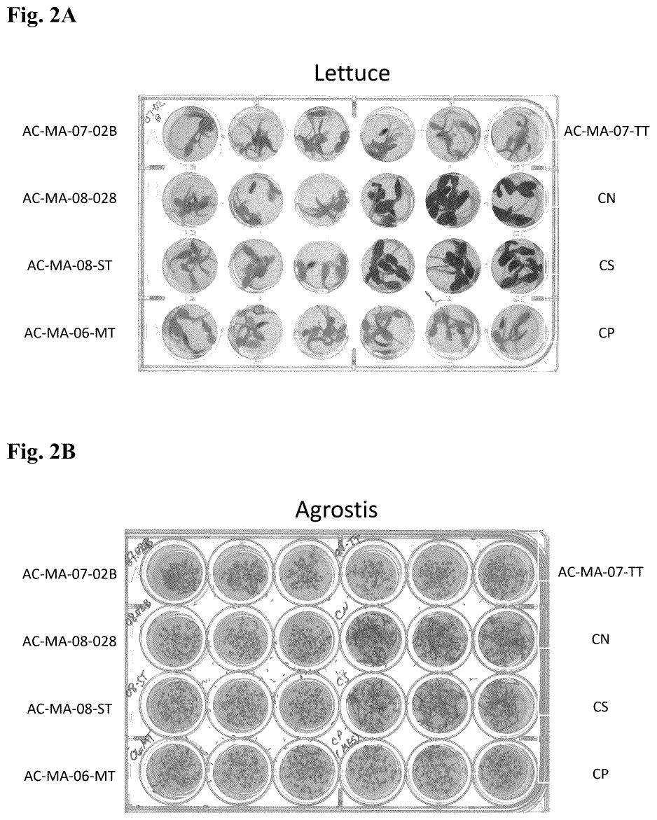

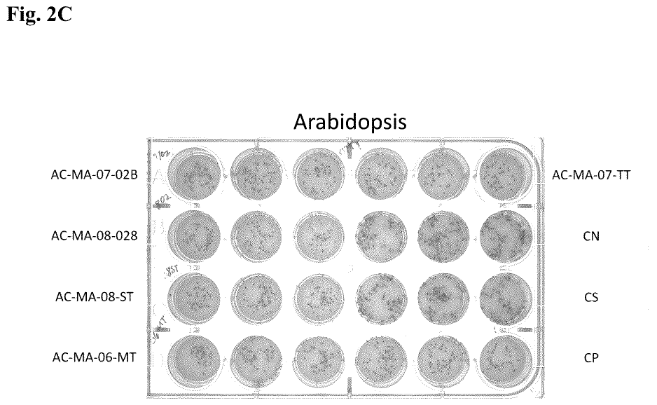

The phytotoxic pre-emergence activity of an exemplary compounds belonging to formula (XIII), compound (XIII-2) and compound (XIII-3) were evaluated in a primary bioassay. The synthetic herbicide acifluorfen was used as positive control. For comparative purposes, water as well as 10% acetone in water were used as negative controls. The seeds of monocot bentgrass (Agrostis stolonfera) and dicots lettuce (Lactuca sativa) and Arabidopsis (Arabidopsis thaliana) were exposed for 7 days to 1000 μM solution of each chemical. As seen in FIG. 2A and FIG. 2B, AC-MA-07-02B (compound (XIII-2), temborione-pelargonic acid conjugate) and AC-MA-08-028 (compound (XIII-3), sulcotrione-pelargonic acid conjugate) significantly impacted growth of the lettuce and the bentgrass seedlings: most of them were chlorotic, some pale green and with developed, shorter leaves compared to negative controls—containing H₂O or 10% acetone. As seen in FIG. 2C, Arabidopsis seedling growth was significantly affected by the AC-MA-07-02B (compound (XIII-2), temborione-pelargonic acid conjugate) and AC-MA-08-028 (compound (XIII-3), sulcotrione-pelargonic acid conjugate). As seen in FIG. 2A, FIG. 2B, and FIG. 2C almost all 7-days-old seedlings exposed to the AC-MA-07-02B (compound (XIII-2), temborione-pelargonic acid conjugate) and AC-MA-08-028 (compound (XIII-3), sulcotrione-pelargonic acid conjugate) developed chlorotic hypocotyls and cotyledons. Chlorosis of treated seedlings is a common symptom of a typical HPPD enzyme inhibition, and the obtained results are an initial confirmation this mode of action for this new compound.

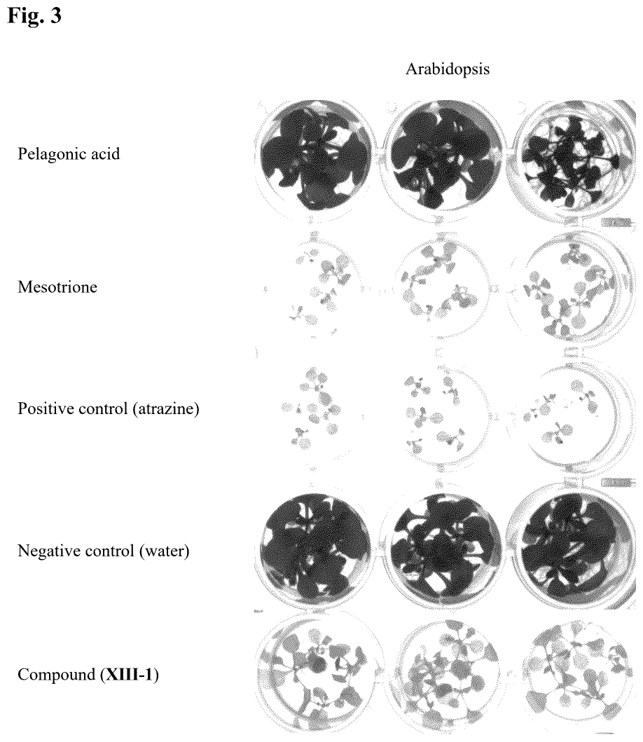

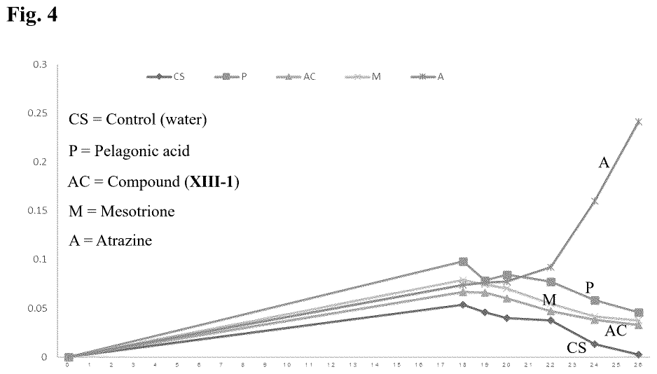

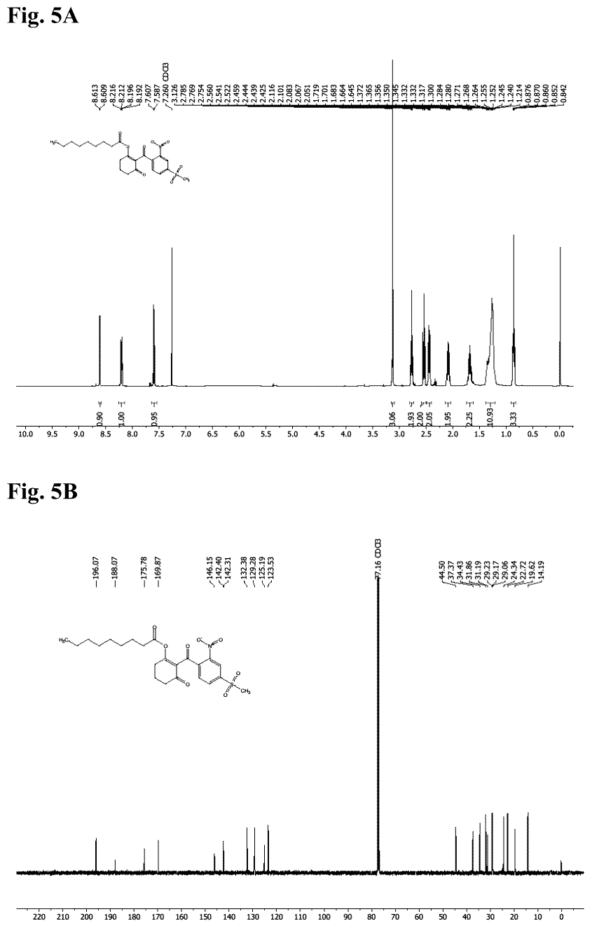

The phytotoxic post-emergence activity of an exemplary compound belonging to formula (XIII), compound (XIII-1) was also evaluated. An image of a scan of 31-day-old Arabidopsis (Arabidopsis thaliana) seedlings exposed when ten-days-old to 100 μM solution of the selected compounds is depicted in FIG. 3. Ten-days old seedlings were submerged in 100 μM solutions for 5 minutes and observed 21 days after treatment. The post-emergence activity results for pelargonic acid, mesotrione, atrazine (positive control), water (negative control), and the compound (XIII-1) for the Arabidopsis (three replicates in each row). The compound (XIII-1) significantly impacted post-emergence growth of the Arabidopsis seedlings: most of them were chlorotic and developed shorter leaves compared to negative controls—containing water.

Next, a detailed assessment of the phytotoxic activity of an exemplary compound belonging to formula (XIII), compound (XIII-1) was done by calculating dose-response curve base on growth rate of duckweed (Lemna paucicostata) frond surface. Mesotrione was used as a positive control. The surface growth of the fronds was measured at seven days from the start point of growth. The dose response curve and the half maximal inhibitory concentration (IC₅₀) were generated in R 4.2.1 software by dre package. As seen in Table 1 below, the calculated IC₅₀ value for the compound (XIII-1) was 6.8 nM while the calculated IC₅₀ value for the control herbicide mesotrione was 0.6 nM. When comparing with the data provided by Michel, A., et al. (2004, Supra), the compound (XIII-1) is more active than the commercial herbicides bentazon, clomazone, glyphosate, glufosinate, asulam, chloropropham, and EPTC in the same bioassay.

The electrolyte leakage (a measure of cellular damage) of 100 μM solutions of the compound (XIII-1), pelargonic acid, mesotrione, atrazine (positive control), and water (negative control) is depicted in FIG. 4.

TABLE 1
Herbicidal activity of DMOA proherbicide in a secondary bioassay.

| Compound | IC₅₀ (μM) |
|---|---|
| XIII-1 | 6.8493e−03 ± 1.5079e−03 |
| Mesotrione | 5.84e−04 ± 8.76e−05 |

The results in this example show that DMOA proherbicides of formula (XIII), where R₄ is independently an alkyl chain having 1 to 20 carbon atoms, including, but not limited to alkyl chain with terminal hydroxy or halogen groups; X₁, X₂, and X₃ are independently H, F, Cl, Br, I, NO₂, SCH₃, SO₂CH₃, or CH₂OR₇, where R₇ is independently any alkyl chain or functionalized alkyl chain; and Z is independently CH or N, have herbicidal activity.

### Example 3

### Synthesis of Keto-Diesters

A general reaction scheme for the preparation of keto-diesters is shown below. The newly prepared keto-diesters have formula (III), where R₁ and R₂ are independently H or an alkyl group; X and Y are independently H, alkyl, NO₂, SO₂Me, CF₃, halide, NH, OH, SH or a derivative thereof, and Z is CH or N.

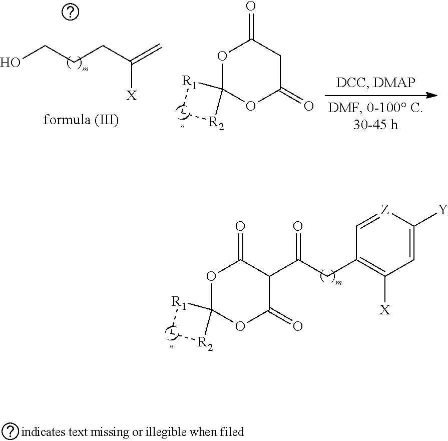

Exemplary keto-diesters were prepared by adding at room temperature (2.0 equiv.) N, N′-dicyclohexylcarbodiimide (DCC) to a solution of aryl carboxylic acid (formula I, 2.0 equiv.) in anhydrous dimethyl formamide (DMF). After 15 minutes, the reaction mixture was cooled to 0° C. and the protected diester (formula II, 1.0 equiv.) was added, followed by N, N′-dimethylamino pyridine (DMAP) (2.0 equiv.) The reaction mixture was heated to 100° C. and stirred at the same temperature for about 30 to 45 hours. At the end of the reaction, the insoluble urea was filtered off and washed with 1,2-dichloroethane. The combined washings were extracted with 1N HCl, and washed with water and brine. After the organic phase was dried over sodium sulfate, the solvent was removed, and the residue was purified by flash-column chromatography.

Exemplary keto-diesters prepared by this method were 5-(2-chloro-4-(methylsulfonyl)benzoyl)-2,2-dimethyl-1,3-dioxane-4,6-dione (formula III-1), 2,2-dimethyl-5-(2-nitro-4-(trifluoromethyl)benzoyl)-1,3-dioxane-4,6-dione (formula III-2), 2,2-dimethyl-5-(4-(methylsulfonyl)-2-nitrobenzoyl)-1,3-dioxane-4,6-dione (formula III-3), 2,2-dimethyl-5-(3-phenylpropanoyl)-1,3-dioxane-4,6-dione (formula III-4), 8-(3-phenylpropanoyl)-6,10-dioxaspiro[4.5]decane-7,9-dione (formula III-5), 5-(3-phenylpropanoyl)-2,2-dipropyl-1,3-dioxane-4,6-dione (formula III-6), 2,2-dimethyl-5-(3-methylbutanoyl)-1,3-dioxane-4,6-dione (formula VII-1) and 8-(3-methylbutanoyl)-6,10-dioxaspiro[4.5]decane-7,9-dione (formula VII-2).

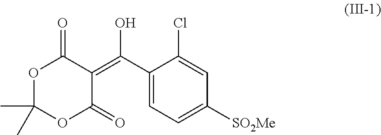 [SMILES: CC1(C)OC(=O)C(=C(O)c2ccc(S(C)(=O)=O)cc2Cl)C(=O)O1]

III-1=5-(2-chloro-4-(methylsulfonyl)benzoyl)-2,2-dimethyl-1,3-dioxane-4,6-dione (R1=R2=Me, X=Cl, Y=SO₂Me and Z=H): ¹H NMR (400 MHz, MeOD) δ 7.88 (d, J=1.7 Hz, 1H), 7.83 (dd, J=8.0, 1.7 Hz, 1H), 7.39 (d, J=8.0 Hz, 1H), 3.15 (s, 3H), 1.68 (s, 6H). HRMS (ESI) m/z calculated for C14H12ClO7S [M-H]− 358.9992, found 359.0036.

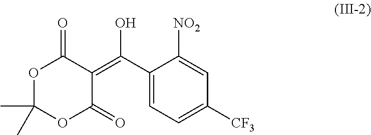 [SMILES: CC1(C)OC(=O)C(=C(O)c2ccc(C(F)(F)F)cc2[N+](=O)[O-])C(=O)O1]

III-2=2,2-dimethyl-5-(2-nitro-4-(trifluoromethyl)benzoyl)-1,3-dioxane-4,6-dione (R1=R2=Me, X=NO₂, Y=CF₃ and Z=H): 1H NMR (400 MHz, MeOD) δ 8.38 (s, 1H), 7.97 (d, J=7.4 Hz, 1H), 7.51 (d, J=7.8 Hz, 1H), 1.66 (s, 6H). HRMS (ESI) m/z calculated for C14H9F3NO7 [M-H]⁻ 360.0331, found 360.0391.

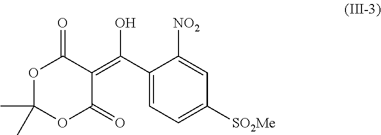 [SMILES: CC1(C)OC(=O)C(=C(O)c2ccc(S(C)(=O)=O)cc2[N+](=O)[O-])C(=O)O1]

III-3=2,2-dimethyl-5-(4-(methylsulfonyl)-2-nitrobenzoyl)-1,3-dioxane-4,6-dione (R1=R2=Me, X=NO₂, Y=SO₂Me and Z=H): H NMR (400 MHz, MeOD) δ 8.61 (d, J=1.7 Hz, 1H), 8.30-8.15 (m, 1H), 7.57 (d, J=8.0 Hz, 1H), 3.22 (s, 3H), 1.67 (s, 6H). HRMS (ESI) m z calculated for C14H12NO₉S [M-H]⁻ 370.0233, found 370.0245.

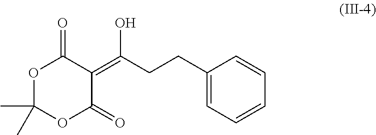 [SMILES: CC1(C)OC(=O)C(=C(O)CCc2ccccc2)C(=O)O1]

III-4=2,2-dimethyl-5-(3-phenylpropanoyl)-1,3-dioxane-4,6-dione (R1=R2=Me, X, Y, Z=H): ¹H NMR (400 MHz, CDCl₃) δ 7.30-7.17 (m, 5H), 3.46-3.28 (m, 2H), 3.01 (dd, J=8.9, 6.7 Hz, 2H), 1.65 (s, 6H). ¹³C NMR (101 MHz, CDCl₃) δ 196.6, 170.6, 160.3, 139.8, 128.7, 128.6, 126.6, 105.0, 92.0, 37.3, 32.1, 26.9.

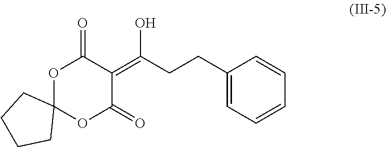 [SMILES: O=C1OC2(CCCC2)OC(=O)C1=C(O)CCc1ccccc1]

III-5=8-(3-phenylpropanoyl)-6,10-dioxaspiro[4.5]decane-7,9-dione (n=1-cyclopentenyl, X, Y, Z=H): ¹H NMR (500 MHz, CDCl₃) δ 15.19 (s, 1H), 7.40-7.10 (m, 5H), 3.49-3.33 (m, 2H), 3.02 (dd, J=8.8, 6.7 Hz, 2H), 2.20-2.00 (m, 4H), 2.00-1.69 (m, 4H). ¹³C NMR (126 MHz, CDCl₃) δ 196.6, 171.3, 161.0, 139.8, 128.7, 128.6, 126.7, 114.3, 92.6, 37.9, 37.3, 32.1, 23.2.

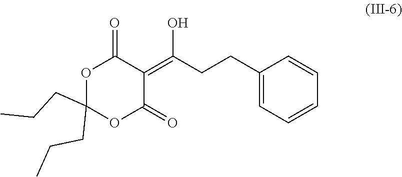 [SMILES: CCCC1(CCC)OC(=O)C(=C(O)CCc2ccccc2)C(=O)O1]

III-6=5-(3-phenylpropanoyl)-2,2-dipropyl-1,3-dioxane-4,6-dione (R1=R2=n-propyl, X, Y, Z=H): ¹H NMR (400 MHz, CDCl₃) δ 15.35 (s, 1H), 7.38-7.13 (m, 5H), 3.51-3.29 (m, 2H), 3.01 (dd, J=8.9, 6.6 Hz, 2H), 1.95-1.71 (m, 4H), 1.59-1.35 (m, 4H), 0.93 (t, J=7.4 Hz, 6H). ¹³C NMR (101 MHz, CDCl₃) δ 196.2, 170.7, 160.3, 139.9, 128.7, 128.6, 126.6, 108.4, 92.0, 39.8, 37.4, 32.0, 16.4, 13.9.

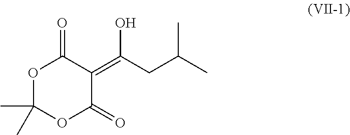 [SMILES: CC(C)CC(O)=C1C(=O)OC(C)(C)OC1=O]

VII-1=2,2-dimethyl-5-(3-methylbutanoyl)-1,3-dioxane-4,6-dione (R1=R2=R3=R4=Me): ¹H NMR (400 MHz, CDCl₃) δ 15.31 (s, 1H), 2.99 (d, J=7.1 Hz, 2H), 2.21 (dp, J=13.6, 6.8 Hz, 1H), 1.74 (s, 6H), 1.03 (s, 3H), 1.01 (s, 3H). ¹³C NMR (101 MHz, CDCl₃) δ 197.7, 170.7, 160.4, 104.8, 92.0, 44.0, 27.5, 26.9, 22.7.

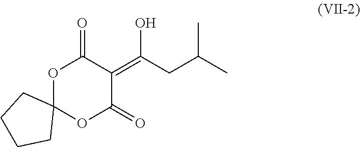 [SMILES: CC(C)CC(O)=C1C(=O)OC2(CCCC2)OC1=O]

VII-2=8-(3-methylbutanoyl)-6,10-dioxaspiro[4.5]decane-7,9-dione (n=1-cyclopentenyl, R3=R4=Me): ¹H NMR (500 MHz, CDCl₃) δ 15.20 (s, 1H), 2.98 (d, J=7.1 Hz, 2H), 2.23-2.13 (m, 5H), 1.85 (ddd, J=7.8, 5.7, 2.5 Hz, 4H), 1.02 (s, 3H), 1.01 (s, 3H). ¹³C NMR (126 MHz, CDCl₃) δ 197.7, 171.4, 161.2, 114.1, 92.6, 43.9, 38.0, 27.6, 23.3, 22.7.

The data presented here shows that keto-diesters of formula (III), or (VII) where R₁ and R₂ are independently H or an alkyl group; X and Y are independently H, alkyl, NO₂, SO₂Me, CF₃, halide, NH, OH, SH or a derivative thereof, and Z is CH or N may be prepared in a one to two step reaction.

### Example 4

### Phytotoxic Activity of Keto-Diesters

The effects of the keto-diesters of the invention in the growth of monocot and dicot seedlings were tested.

The phytotoxic activity of compounds belonging to formula III (III-1, III-2, and III-3) was evaluated in a primary bioassay. The synthetic triketone herbicides mesotrione and sulcotrione were used as positive controls. For comparative purposes, water as well as 10% acetone in water were used as negative controls. The seeds of monocot bentgrass (Agrostis stolonifera) and dicot Arabidopsis (Arabidopsis thaliana) were exposed for 7 days to 1000 μM solution of each chemical. The compound III-2 significantly impacted growth of the bentgrass seedlings: most of them were chlorotic, some pale green and with developed, shorter leaves compared to negative controls—containing H₂O or 10% acetone. The seedlings growing with compounds 111-3 and 111-1 did not develop chlorosis, although growth of some of them was reduced. Arabidopsis seedling growth was significantly affected by all of the tested compounds. As seen in FIG. 7A, almost all 7-days-old seedlings exposed to any one of three new compounds developed chlorotic hypocotyls and cotyledons. Chlorosis of treated seedlings is a common symptom of a typical HPPD enzyme inhibition, and the obtained results are an initial confirmation this mode of action for these new compounds.

Next, a detailed assessment of the phytotoxic activity of the potential herbicide candidates was done by calculating dose-response curve base on growth rate of duckweed (Lemna paucicostata) frond surface. Mesotrione and sulcotrione were used as positive controls and water and 1% acetone in water were used as negative controls. As seen in Table 2 below, III-2 was the most phytotoxic tested compound with an IC₅₀ of 1.9 μM while the remaining two chemicals were less potent: III-1 with 192.9 μM and III-3 with 46 μM. When comparing with the data provided by Michel, A., et al. (2004, Supra), the most active of the newly prepared compounds (III-2) is more active than the commercial herbicides bentazon, clomazone, glyphosate, glufosinate, asulam, chloropropham, and EPTC in the same bioassay.

The phytotoxic activity of compounds belonging to formula (III), compounds (III-4), (III-5), and (III-6) was evaluated in a primary bioassay. The synthetic triketone herbicides mesotrione and sulcotrione were used as positive controls. For comparative purposes, water as well as 10% acetone in water were used as negative controls. The seeds of monocot bentgrass (Agrostis stolonifera) and dicot Arabidopsis (Arabidopsis thaliana) were exposed for 7 days to 1000 μM solution of each chemical. Novel compounds belonging to (formula V) significantly impacted germination and growth of the bentgrass seedlings: most of them were chlorotic and developed shorter leaves compared to negative controls—containing H₂O or 10% acetone. As seen in FIG. 7B, almost all Arabidopsis seeds exposed to any of the three new compounds did not germinate, or showed emerging root tip without developed hypocotyls and cotyledons.

Next, a detailed assessment of the phytotoxic activity of the potential herbicide candidates was done by calculating dose-response curves base on growth rate of duckweed (Lemna paucicostata) frond surface. Mesotrione and sulcotrione were used as positive controls and water and 1% acetone in water were used as negative controls. As seen in Table 2 below, compounds III-4, III-6, and III-5 were the most phytotoxic tested compounds with an IC₅₀ of 13.2 μM, 2.6 μM and 17.9 μM. Again, when comparing with the data provided by Michel, A., et al. (2004, Supra), the newly prepared compound with formula III-6 is more active than the commercial herbicides bentazon, clomazone, glyphosate, glufosinate, asulam, chloropropham, or EPTC in the same bioassay.

The phytotoxic activity of compounds belonging to formula (VII) (compounds (VII-1) and (VII-2)) was evaluated in a primary bioassay. The synthetic triketone herbicides mesotrione and sulcotrione were used as positive controls. For comparative purposes, water as well as 10% acetone in water were used as negative controls. The seeds of monocot bentgrass (Agrostis stolonfera) and dicot Arabidopsis (Arabidopsis thaliana) were exposed for 7 days to 1000 μM solution of each chemical. Novel compounds belonging to the formula (VII) significantly impacted bentgrass and Arabidopsis seed germination and growth of the seedlings: most of them were chlorotic and developed shorter leaves when compared to negative controls—containing H₂O or 10% acetone. As seen in FIG. 7B, almost all the seeds did not germinate or showed emerging root tip without developed hypocotyls and coleoptile. Chlorosis of treated seedlings is a common symptom of a typical HPPD enzyme inhibition, and the obtained results are an initial confirmation this mode of action for these new compounds.

Next, a detailed assessment of the phytotoxic activity of the potential herbicide candidates was done by calculating dose-response curve based on the growth rate of duckweed (Lemna paucicostata) frond surface. Mesotrione and sulcotrione were used as positive controls and water and 1% acetone in water were used as negative controls. As seen in Table 2 below, both the tested compounds of formula (VII) displayed significant phytotoxic activity with an IC₅₀ of 1.14 μM for VII-1 and 3.3 μM for VII-2. Again, when comparing with the data provided by Michel, A., et al. (2004, Supra), most of the newly prepared compounds with formula VII are more active than the commercial herbicides bentazon, clomazone, glyphosate, glufosinate, asulam, chloropropham, or EPTC in the same bioassay.

TABLE 2
Herbicidal activity of novel herbicides in a secondary bioassay.

| Compound | IC₅₀ (μM) |
|---|---|
| III-1 | 46 ± 7.7 |
| III-2 | 1.9 ± 0.46 |
| III-3 | 192.9 ± 35.5 |
| III-4 | 13.2 ± 2.65 |
| III-5 | 17.9 ± 7.4 |
| III-6 | 2.6 ± 1.1 |
| VII-1 | 1.14 ± 0.23 |
| VII-2 | 3.3 ± 1.31 |
| Mesotrione | 5.84e−04 ± 8.76e−05 |
| Sulcotrione | 2.79e−04 ± 4.58e−05 |

The results in this example show that the keto-diesters of formula (III), (V), or (VII) where R₁ and R₂ are independently H or an alkyl group; X and Y are independently H, alkyl, NO₂, SO2Me, CF₃, halide, NH, OH, SH or a derivative thereof, and Z is CH or N, have herbicidal activity.

We claim:

1. A compound having formula (IX) 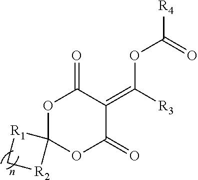
or a herbicidally-effective salt thereof,
wherein:
n is independently 0, 1, 2, 3, 4, or 5;
R₁ and R₂ are independently H or an alkyl group;
R₄ is independently an alkyl chain having 1 to 20 carbon atoms; and
R₃ is independently 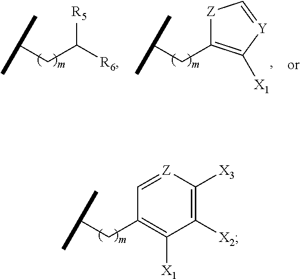
wherein:
m is independently 0, 1, 2, 3, 4, or 5;
R₅ and R₆ are independently H, alkyl, aryl, heteroaryl, alkyl aryl, or alkyl heteroaryl;
X₁, X₂, and X₃ are independently H, F, Cl, Br, I, NO₂, SCH₃, SO₂CH₃, or CH₂OR₇, wherein
R₇ is independently any alkyl chain or halogenated alkyl chain;
Y is independently NH, 0, or S; and
Z is independently CH or N.

2. A composition comprising the compound of claim 1, further comprising a carrier and/or a surfactant.

3. The composition of claim 2, further comprising at least one additional biologically-active compound.

4. The composition of claim 3, wherein the at least one additional biologically-active compound is a fungicide, an insecticide, a nematicide, a bactericide, or an acaricide.

5. A method for preparing the compound of claim 1, the method comprising condensation of at least one tautomer of a HPPD inhibitor of formula (VIII) 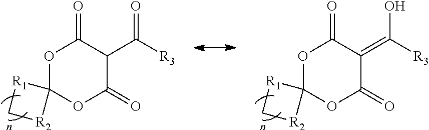
wherein:
n is independently 0, 1, 2, 3, 4, or 5;
R₁ and R₂ are independently H or an alkyl group; and
R₃ is independently 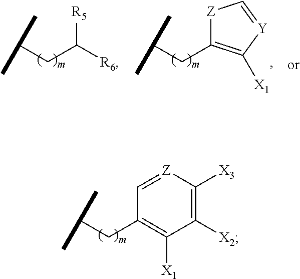
wherein:
m is independently 0, 1, 2, 3, 4, or 5;
R₅ and R₆ are independently H, alkyl, aryl, heteroaryl, alkyl aryl, or alkyl heteroaryl;
X₁, X₂, and X₃ are independently H, F, Cl, Br, I, NO₂, SCH₃, SO₂CH₃, or CH₂OR₇, wherein
R₇ is independently any alkyl chain or halogenated alkyl chain;
Y is independently NH, O, or S; and
Z is independently CH or N;
with at least one fatty acid having 6 to 20 carbon atoms in the presence of a base and an organic solvent in a single pot reaction to prepare a novel herbicide.

6. The method of claim 5, wherein the base is triethyl amine and the organic solvent is 1,2-dichloroethane.

7. A method for controlling undesired vegetation, the method comprising contacting the vegetation or its environment with an effective amount of a compound of claim 1.

8. A method for controlling undesired vegetation, the method comprising contacting the vegetation or its environment with an effective amount of a composition of claim 2.

9. A method for controlling undesired vegetation, the method comprising contacting the vegetation or its environment with an effective amount of a composition of claim 3.

10. A method for controlling undesired vegetation, the method comprising contacting the vegetation or its environment with an effective amount of a composition of claim 4.

11. A compound having at least one tautomer of formula (XI) 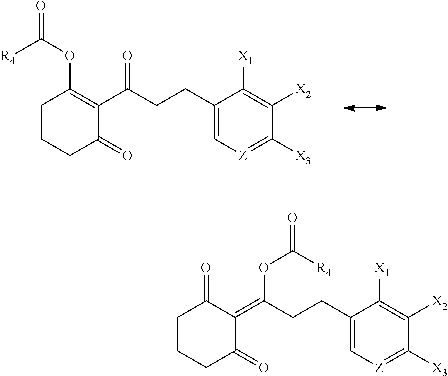
or a herbicidally-effective salt thereof,
wherein:
R₄ is independently an alkyl chain having 1 to 20 carbon atoms;
X₁, X₂, and X₃ are independently H, F, Cl, Br, I, NO₂, SCH₃, SO₂CH₃, or CH₂OR₇, wherein
R₇ is independently any alkyl chain or functionalized alkyl chain; and
Z is independently CH or N.

12. A composition comprising the compound of claim 11, further comprising a carrier and/or a surfactant.

13. The composition of claim 12, further comprising at least one additional biologically-active compound.

14. The composition of claim 13, wherein the at least one additional biologically-active compound is a fungicide, an insecticide, a nematicide, a bactericide, or an acaricide.

15. A method for preparing the compound of claim 11, the method comprising condensation of at least one tautomer of a HPPD inhibitor of formula (X) 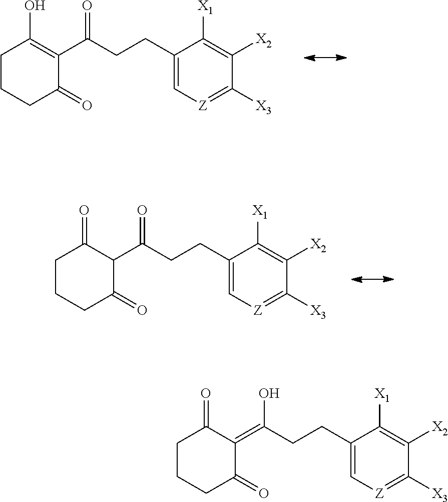
wherein:
X₁, X₂, and X₃ are independently H, F, Cl, Br, I, NO₂, SCH₃, SO₂CH₃, or CH₂OR₇, wherein
R₇ is independently any alkyl chain or functionalized alkyl chain; and
Z is independently CH or N;
with at least one fatty acid having 6 to 20 carbon atoms in the presence of a base and an organic solvent in a single pot reaction to prepare a novel herbicide.

16. The method of claim 15, wherein the base is triethyl amine and the organic solvent is 1,2-dichloroethane.

17. A method for controlling undesired vegetation, the method comprising contacting the vegetation or its environment with an effective amount of a compound of claim 11.

18. A method for controlling undesired vegetation, the method comprising contacting the vegetation or its environment with an effective amount of a composition of claim 12.

19. A method for controlling undesired vegetation, the method comprising contacting the vegetation or its environment with an effective amount of a composition of claim 13.

20. A method for controlling undesired vegetation, the method comprising contacting the vegetation or its environment with an effective amount of a composition of claim 14.

21. A compound having at least one tautomer of formula (XIII) 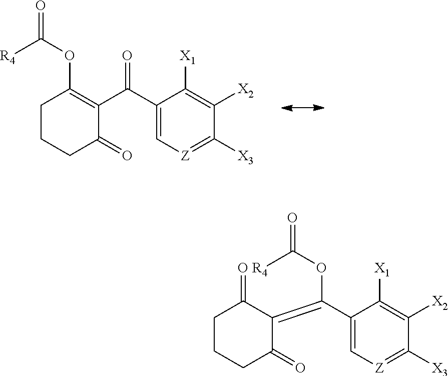
or a herbicidally-effective salt thereof,
wherein:
R₄ is independently an alkyl chain having 1 to 20 carbon atoms;
X₁, X₂, and X₃ are independently H, F, Cl, Br, I, NO₂, SCH₃, SO₂CH₃, or CH₂OR₇, wherein
R₇ is independently any alkyl chain or functionalized alkyl chain; and
Z is independently N; or
wherein:
R₄ is independently an alkyl chain having 7 to 20 carbon atoms;
X₁, X₂, and X₃ are independently H, F, Cl, Br, I, NO₂, SCH₃, SO₂CH₃, or CH₂OR₇, wherein
R₇ is independently any alkyl chain or functionalized alkyl chain; and
Z is independently CH or N; or
wherein:
R₄ is independently an alkyl chain having 1 to 20 carbon atoms;
X₁, X₂, and X₃ are independently H, F, Cl, Br, I, NO₂, SCH₃, SO₂CH₃, or CH₂OR₇, wherein
R₇ is independently any alkyl chain or functionalized alkyl chain, and wherein at least one of X₁, X₂, and X₃ is CH₂OR₇; and
Z is independently CH or N.

22. A composition comprising the compound of claim 21, further comprising a carrier and/or a surfactant.

23. The composition of claim 22, further comprising at least one additional biologically-active compound.

24. The composition of claim 23, wherein the at least one additional biologically-active compound is a fungicide, an insecticide, a nematicide, a bactericide, or an acaricide.

25. A method for preparing the compound of claim 21, the method comprising condensation of at least one tautomer of a HPPD inhibitor of formula (XII) 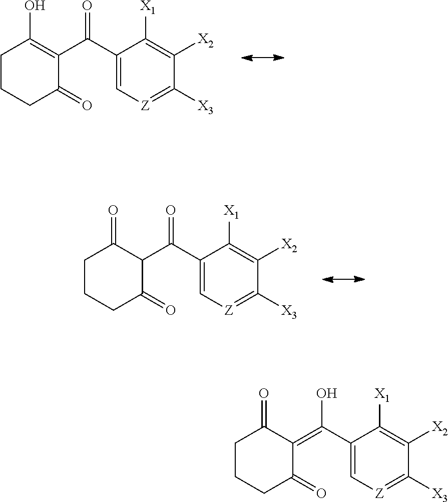
wherein:
X₁, X₂, and X₃ are independently H, F, Cl, Br, I, NO₂, SCH₃, SO₂CH₃, or CH₂OR₇, wherein
R₇ is independently any alkyl chain or functionalized alkyl chain; and
Z is independently CH or N;
with at least one fatty acid or functionalized fatty acid having 6 to 20 carbon atoms in the presence of a base and a non-protic organic solvent in a single pot reaction to prepare a novel proherbicide with two independent mechanisms of action.

26. The method of claim 25, wherein the base is triethyl amine and the aprotic organic solvent is 1,2-dichloroethane.

27. A method for controlling undesired vegetation, the method comprising contacting the vegetation or its environment with an effective amount of a compound of claim 21.

28. A method for controlling undesired vegetation, the method comprising contacting the vegetation or its environment with an effective amount of a composition of claim 22.

29. A method for controlling undesired vegetation, the method comprising contacting the vegetation or its environment with an effective amount of a composition of claim 23.

30. A method for controlling undesired vegetation, the method comprising contacting the vegetation or its environment with an effective amount of a composition of claim 24.

31. A method of producing a novel proherbicide comprising condensation of a HPPD inhibitor with at least one fatty acid having 1-20 carbon atoms in the presence of a base and an organic solvent in a single pot reaction, wherein the HPPD inhibitor is selected from the group consisting of bicyclopyrone, fenquinotrione, nitisinone, 4-(2,6-diethyl-4-methylphenyl)-5-hydroxy-2,6-dimethyl-3(2H)-pyridazinone (PubChem CID 54683778), 4-(4-fluorophenyl)-6-[(2-hydroxy-6-oxo-1-cyclohexen-1-yl)carbonyl]-2-methyl-1,2,4-triazine-3,5(2H,4H)-dione (PubChem CID 71566698), 2-[hydroxy-[2-(3-methoxyphenyl)-1-(3-methoxypropyl)-6-oxopyrimidin-5-yl]methylidene]cyclohexane-1,3-dione (PubChem CID 90022616), and combinations thereof.

32. The method of claim 31, wherein the base is triethyl amine and the organic solvent is 1,2-dichloroethane.

33. A composition comprising a novel proherbicide prepared by the method of claim 31.
